(19) United States
(12) Patent Application Publication
(10) Pub. No.: US 2024/0368298 A1
(43) Pub. Date: Nov. 7, 2024

(54) GENE SIGNATURES FOR MONITORING ACUTE REJECTION AND METHODS OF USING SAME

(71) Applicant: Duke University, Durham, NC (US)

(72) Inventors: Eileen Chambers, Durham, NC (US); Qing Cheng, Durham, NC (US); Allan Kirk, Durham, NC (US)

(73) Assignee: Duke University, Durham, NC (US)

(21) Appl. No.: 18/773,676

(22) Filed: Jul. 16, 2024

Related U.S. Application Data
(63) Continuation of application No. 17/245,794, filed on Apr. 30, 2021, now Pat. No. 12,084,509.
(60) Provisional application No. 63/017,695, filed on Apr. 30, 2020.

(51) Int. Cl. C07K 16/28; A61K 31/573; A61K 45/06; C12Q 1/6876

(52) U.S. Cl. CPC C07K 16/2887; A61K 31/573; A61K 45/06; C07K 16/2809; C07K 16/2893; C12Q 1/6876

(57) ABSTRACT

The present disclosure provides methods and compositions for detecting and treating acute transplant rejection.

### PRIOR RELATED APPLICATION

This application is a continuation of U.S. application Ser. No. 17/245,794, filed on Apr. 30, 2021, which claims the benefit of and priority to U.S. Provisional Application No. 63/017,695 filed on Apr. 30, 2020, which are hereby incorporated by reference in their entirety.

### FIELD

This disclosure describes compositions and methods for monitor and treating acute transplant rejection.

### REFERENCE TO A SEQUENCE LISTING SUBMITTED AS A TEXT FILE VIA EFS-WEB

The official copy of the sequence listing is submitted electronically via EFS-Web as an ASCII formatted sequence listing with a file named 106707-1244770_seqlist.txt, created on Apr. 30, 2021, and having a size of 109 kb, and is filed concurrently with the specification. The sequence listing contained in this ASCII formatted document is part of the specification and is herein incorporated by reference in its entirety.

### BACKGROUND

Despite advancements in clinical care for kidney transplant patients, long term outcomes remain sub-optimal. The reported incidence of acute rejection (AR)—including antibody mediated rejection (ABMR) and T cell mediated rejection (TCMR)—in the first year after transplantation varies depending on the immunosuppression utilized. It is typically higher with steroid and calcineurin inhibitor minimization or Belatacept-based regimens, though these regimens are often preferred for younger recipients as the reduction in long-term side effects is thought to offset the increased risk of early, treatable AR. Regardless, AR has been associated with decreased long-term allograft survival in both pediatric and adult studies. Additionally, TCMR has been correlated with formation of de novo donor specific antibody (dnDSA) which is strongly associated with premature allograft loss. Finally, AR is often associated with inflammation within areas of interstitial fibrosis and tubal atrophy (i-IFTA) at one year that is also correlated with decreased allograft survival. Immune monitoring to detect AR allows for early intervention and decreased graft damage, but diagnostic methods, particularly those relying on molecular signatures, are lacking, as these methods can be influenced by differences in the immunosuppressive strategies used, and these differences are non-uniformly distributed by recipient age. There is a need for compositions and methods for detection of both TCMR and ABMR in pediatric and adult patients, regardless of immunosuppression regimen utilized.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

The present disclosure is based, in part, on the findings by the inventors of a unique age-independent gene signature for acute transplant rejections that is effective across a broad array of immunosuppressive regimens.

Provided herein is a method for treating acute rejection (AR) of a transplant in a subject comprising: (a) measuring expression levels of two or more genes selected from the group consisting of DIP2C, ENOSF1, FBXO21, KCTD6, PDXDC1, REXO2, HLA-E, and RAB31 in a biological sample from a subject having a transplant, wherein differential expression of two or more genes selected from the group consisting of DIP2C, ENOSF1, FBX021, KCTD6, PDXDC1, REXO2, HLA-E, and RAB31, as compared to control, indicates the subject has an increased likelihood of AR of the transplant; and (b) administering an effective amount of corticosteroid or antibody therapy to the subject.

In some embodiments, the expression levels of DIP2C, ENOSF1, FBXO21, KCTD6, PDXDC1, REXO2, HLA-E, and RAB31 are measured. In some embodiments, differential expression results in a genetic signature wherein DIP2C, ENOSF1, FBX021, KCTD6, PDXDC1 and REXO2 are downregulated, and wherein HLA-E and RAB31 are upregulated.

In some embodiments, mRNA expression levels are measured. In some embodiments, protein expression levels are measured.

In some embodiments, the acute cellular rejection is T cell-mediated rejection (TCMR) or antibody-mediated rejection (ABMR).

In some embodiments, the transplant is a kidney transplant. In some embodiments, the subject is an adult. In some embodiments, the subject is a pediatric subject.

In some embodiments, the antibody is selected from the group consisting of a lymphocyte-depleting antibody, an anti-thymoglobin antibody, an anti-CD52 antibody, for example, alemtuzumab, and an anti-CD3 antibody.

In some embodiments, the method further comprises performing a biopsy on transplant tissue from the subject. In some embodiments, the method further comprises administering plasma exchange therapy, intravenous immunoglobulin (Ig) therapy, anti-IL-6 therapy, or a proteosomal inhibitor, if the biopsy shows antibody-mediated damage in the subject. In some embodiments, intravenous Ig therapy, an anti-IL-6 therapy, or a proteosomal inhibitor is administered in combination with rituximab.

Also provided is a kit comprising: (a) primers or probes for detection of two or more genes selected from the group consisting of DIP2C, ENOSF1, FBX021, KCTD6, PDXDC1, REXO2, HLA-E, and RAB31.

### DESCRIPTION OF THE FIGURES

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIGS. 1A-ID are PCA plots of batch effect normalization, according to certain embodiments of this disclosure. FIG. 1A and FIG. 1C are PCA plots before and after normalization among renal samples. FIG. 1B and FIG. 1D are PCA plots before and after normalization among blood cell samples. These plots show the gene expression profiles of the samples plotted on the first two principal components. Each point represents a sample, and samples from the same data set have the same color. These figures demonstrate there are no batch effects.

FIGS. 2A-2D are discordant gene expression profiles between adult and pediatric cases with renal allograft rejection according to certain embodiments of this disclosure. FIG. 2A shows PCA of TCMR, CAN and borderline associated genes reveal significant differences in TCMR and CAN gene profiles between adult and pediatric patients, but not in borderline samples. The upper left panel shows PCA of TCMR using first two principle components (PC1 and PC2) of differentially expressed probe sets between TCMR and STA. The bottom left panel shows PCA of borderline samples using first two principle components (PC1 and PC2) of differential expressed probe sets between borderline and STA. The upper right panel shows PCA of CAN using the first two principle components (PC1 and PC2) of differentially expressed probe sets between CAN and STA. The bottom right panel shows sample distribution defined using PC1 of TCMR associated probe sets and PC1 of CAN associated probe sets, colored by sample type. FIG. 2B shows PCA of TCMR, CAN, and borderline rejection after removal of Age-related differentially expressed genes. FIG. 2C shows 3D PCA of TCMR, CAN and borderline associated genes prior to removing differentially expressed genes between children and adults. FIG. 2D shows 3D PCA of TCMR, CAN and borderline associated genes after removing differentially expressed genes between children and adults yields.

FIG. 3 are schematics showing a workflow for developing age-independent signature of AR using both renal and blood cell samples according to certain embodiments of this disclosure. Part A across the top of the drawing shows a description of clinical samples used in creation of an initial 90 probe set signature. Part B across the middle of the drawing illustrates a workflow showing the multiple comparisons made to identify the initial 90 probe sets. Part C across the bottom of the drawing shows a workflow for identifying early AR predictor.

FIGS. 4A-4C are graphs showing an age-independent signature AR in renal parenchymal and peripheral blood samples according to certain embodiments of this disclosure. (A) 90-probe set model for the identification of AR event 5 years post-transplant using renal tissue samples. (B) 90-probe sets model for the identification of AR event 5 years post-transplant using blood cells. (C) AR-free survival between high and low AR risk groups defined by renal AR signature. ROC curves are plotted with AUCs noted (left panel). Logistic regression analysis was performed using non-parametric Mann-Whitney U test, lines represent median and interquartile range.

FIGS. 5A-5B are graphs showing validation of AR signature in vitro and in silico using independent data sets according to certain embodiments of this disclosure. FIG. 5A shows a ROC curve of 8-gene indentifier of AR event within 1 year after kidney transplant using Duke blood cell samples (training set). FIG. 5B shows a ROC curve of 8-gene identifier of AR event within 1 year after kidney transplant by in silico analysis (testing set). Logistic regression analysis was performed using non-parametric Mann-Whitney U test, lines represent median and interquartile range.

FIG. 6 is a schematic showing a novel gene network using STRING v11 that included the pathways noted in Example 1 in accordance with an embodiment of the present disclosure.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Organ transplantation or the transfer of an organ from one human to another continues to rise throughout the world as the treatment of choice when an organ is irreversibly damaged or organ function is severely impaired. Organ transplantation is not without complications, not only from the transplant surgery itself, but also from the transplant recipient's own immune system and this process, if it happens suddenly, is called acute rejection. For example, when acute rejection of a kidney transplant occurs, it manifests itself by a sudden deterioration in kidney transplant function. About 30 percent of transplant recipients experience an episode of acute rejection. Acute rejection can be associated with reduction in the one-year survival rate of kidney grafts from a deceased donor of about 20 percent, and the projected half-life is about four years shorter in patients who have had an episode of acute rejection compared to patients who have not had an episode of acute rejection.

Sometimes, acute rejection can result from the activation of recipient's T cells and/or B cells. The rejection primarily due to T cells is classified as T cell mediated acute rejection or acute cellular rejection (ACR) and the rejection in which B cells are primarily responsible is classified as antibody mediated acute rejection (AMR). Often times, acute rejection of either type can result in the complete loss of transplant function and transplant failure.

An increase in the level of serum creatinine, a clinically used measure of kidney function, is often the first clinical indicator of acute rejection, and is currently the best surrogate marker of acute rejection of either type. However, this biomarker lacks sensitivity and specificity because graft dysfunction can occur due to non-immunological causes. Further, numerous therapies are used to prevent AR that differ by center and recipient age. This variability confounds diagnostic methods.

Currently, acute rejection is diagnosed by performing an invasive core needle biopsy procedure, which obtains a biopsy of the kidney graft. The histological features in the allograft biopsy tissues are then observed. However, this invasive biopsy procedure is associated with complications such as bleeding, arteriovenous fistula, graft loss, and, in severe cases, even death. Development of a noninvasive and/or minimally invasive test either to anticipate an episode of acute rejection or to diagnose acute rejection without performing the transplant biopsy procedure is a major and an unmet goal in organ transplantation.

Provided herein is a age-independent gene signature for AR effective across a broad array of immunosuppressive regimens. As described in the Examples, kidney transplant biopsy and peripheral blood gene expression profiles from twelve independent public datasets were compiled. After removing genes differentially expressed in pediatric and adults, gene expression profiles from biopsy and peripheral blood samples of patients with AR were compared to those who were stable (STA), using Mann-Whitney U Tests with validation in independent testing datasets. A novel age-independent gene network that identified AR from both kidney and blood samples, irrespective of immunosuppression regimen or recipient age, was identified. This signature was confirmed in pediatric and adult patients.

### Methods

Provided herein is a method for treating acute rejection (AR) of a transplant in a subject comprising, consisting essentially of, or consisting of: (a) measuring expression levels of two or more genes selected from the group consisting of DIP2C, ENOSF1, FBXO21, KCTD6, PDXDC1, REXO2, HLA-E, and RAB31 in a biological sample from a subject having a transplant, wherein differential expression of two or more genes selected from the group consisting of DIP2C, ENOSF1, FBX021, KCTD6, PDXDC1, REXO2, HLA-E, and RAB31, as compared to control, indicates the subject has an increased likelihood of AR of the transplant; and (b) administering an effective amount of a corticosteroid or antibody therapy to the subject.

In some embodiments, the expression levels of two or more, three or more, four or more, five or more, six or more, or seven or more genes selected from the group consisting of DIP2C, ENOSF1, FBXO21, KCTD6, PDXDC1, REXO2, HLA-E, and RAB31 are measured. In some embodiments, the expression levels of DIP2C, ENOSF1, FBXO21, KCTD6, PDXDC1, REXO2, HLA-E, and RAB31 are measured. In some embodiments, differential expression results in a genetic signature wherein DIP2C, ENOSF1, FBXO21, KCTD6, PDXDC1 and REXO2 are downregulated, and wherein HLA-E and RAB31 are upregulated. In some embodiments, differential expression results in a genetic signature wherein DIP2C, ENOSF1, FBX021, KCTD6, PDXDC1 and REXO2 are downregulated, and wherein HLA-E and RAB31 are upregulated.

Also provided is a method for treating AR of a transplant in a subject comprising, consisting essentially of, or consisting of: administering an effective amount of a corticosteroid or antibody therapy to a subject having differential expression of two or more genes selected from the group consisting of DIP2C, ENOSF1, FBXO21, KCTD6, PDXDC1, REXO2, HLA-E, and RAB31, as compared to control. In some embodiments, the subject has differential expression of two or more, three or more, four or more, five or more, six or more, or seven or more genes selected from the group consisting of DIP2C, ENOSF1, FBXO21, KCTD6, PDXDC1, REXO2, HLA-E, and RAB31. In some embodiments, differential expression results in a genetic signature wherein DIP2C, ENOSF1, FBX021, KCTD6, PDXDC1, and/or REXO2 are downregulated, and wherein HLA-E and/or RAB31 are upregulated. In some embodiments, differential expression results in a genetic signature wherein DIP2C, ENOSF1, FBXO21, KCTD6, PDXDC1 and REXO2 are downregulated, and wherein HLA-E and RAB31 are upregulated.

Also provided is a method for identifying an increased likelihood of acute rejection (AR) of a transplant in a subject comprising, consisting essentially of, or consisting of: measuring expression levels of two or more genes selected from the group consisting of DIP2C, ENOSF1, FBXO21, KCTD6, PDXDC1, REXO2, HLA-E, and RAB31 in a biological sample from a subject having a transplant, wherein differential expression of two or more of genes selected from the group consisting of DIP2C, ENOSF1, FBXO21, KCTD6, PDXDC1, REXO2, HLA-E, and RAB31, as compared to control, indicates the subject has an increased likelihood of AR of the transplant.

In some embodiments, the expression levels of two or more, three or more, four or more, five or more, six or more, or seven or more genes selected from the group consisting of DIP2C, ENOSF1, FBXO21, KCTD6, PDXDC1, REXO2, HLA-E, and RAB31 are measured to determine the likelihood of AR of a transplant in the subject. In some embodiments, the expression levels of DIP2C, ENOSF1, FBXO21, KCTD6, PDXDC1, REXO2, HLA-E, and RAB31 are measured to determine the likelihood of AR of a transplant in the subject. In some embodiments, differential expression results in a genetic signature wherein DIP2C, ENOSF1, FBX021, KCTD6, PDXDC1 and/or REXO2 are downregulated, and wherein HLA-E and/or RAB31 are upregulated. In some embodiments, differential expression results in a genetic signature wherein DIP2C, ENOSF1, FBXO21, KCTD6, PDXDC1 and REXO2 are downregulated, and wherein HLA-E and RAB31 are upregulated.

As used throughout, by “subject” is meant an individual. The term “subject” and “patient” are used interchangeably herein and refer to both human and nonhuman animals The subject can be an adult subject or a pediatric subject. Adult subjects include subjects older than eighteen years of age. Pediatric subjects include subjects ranging in age from birth to eighteen years of age. Preferably, the subject is an animal, for example, a mammal such as a primate, and, more preferably, a human. Non-human primates are subjects as well. The term subject includes cats, dogs, reptiles, amphibians, livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical formulations are contemplated herein.

As used herein, a “biological sample” includes any sample obtained from a subject. A sample may contain tissue, cells, proteins, nucleic acids or other cellular matter. A sample may also be the liquid phase of a body fluid from which sedimentary materials have been substantially removed. Exemplary samples include, but are not limited to, blood samples containing peripheral blood mononuclear cells (PBMCs), plasma, urine samples containing urinary cells, fluid “supernatant” that is substantially free of cells, a sample of bronchoalveolar lavage fluid, a sample of bile, pleural fluid or peritoneal fluid, or any other fluid secreted or excreted by a normally or abnormally functioning allograft, or any other fluid resulting from exudation or transudation through an allograft or in anatomic proximity to an allograft, or any fluid in fluid communication with the allograft. A sample may also be obtained from essentially any body fluid including: blood (including peripheral blood), lymphatic fluid, sweat, peritoneal fluid, pleural fluid, bronchoalveolar lavage fluid, pericardial fluid, gastrointestinal juice, bile, urine, feces, tissue fluid or swelling fluid, joint fluid, cerebrospinal fluid, or any other named or unnamed fluid gathered from the anatomic area in proximity to the allograft or gathered from a fluid conduit in fluid communication with the allograft. For example, the sample can be a urinary cell sample. A “post-transplantation sample” refers to a sample obtained from a subject after the transplantation has been performed.

As used herein, the term “transplantation” refers to the process of taking a cell, tissue, or organ, called a “transplant” or “graft” from one individual and placing it or them into a (usually) different individual. The individual who provides the transplant is called the “donor” and the individual who received the transplant is called the “recipient” (or “host”). An organ, or graft, transplanted between two genetically different individuals of the same species is called an “allograft.” A graft transplanted between individuals of different species can be referred to as a “xenograft.”

As used herein, “transplant rejection” refers to a functional and structural deterioration of the organ due to an active immune response expressed by the recipient, and independent of non-immunologic causes of organ dysfunction. Acute transplant rejection (AR) can result from the activation of recipient's T cells and/or B cells. A rejection primarily due to T cells is classified as T cell mediated acute rejection (TCR) (also known as acute cellular rejection (ACR)), and a rejection in which B cells are primarily responsible is classified as antibody mediated acute rejection (AMR). In some embodiments, the methods and compositions provided can detect and/or predict acute cellular rejection.

As used throughout, the term “gene” refers to a nucleic acid, DNA or RNA, involved in producing or encoding a polypeptide. It may include non-coding regions preceding and following the coding region (leader and trailer) as well as intervening sequences (introns) between individual coding segments (exons). As used throughout, the term “nucleic acid” or “nucleotide” refers to deoxyribonucleic acids (DNA) or ribonucleic acids (RNA) and polymers thereof in either single- or double-stranded form. It is understood that when a DNA sequence is described, its corresponding RNA is also described, wherein thymidine is represented as uridine. Unless otherwise indicated, a particular nucleic acid sequence also implicitly encompasses modified variants thereof, alleles, orthologs, SNPs, and complementary sequences as well as the sequence explicitly indicated.

Disco-interacting protein 2 homolog C (DIP2C) is a protein that shares a strong similarity with a Drosophila protein which interacts with transcription factor disco and is expressed in the nervous system. SEQ ID NO: 1, set forth under GenBAnk Accession No. NM_014974.3, is a representative nucleotide sequence encoding DIP2C. The nucleotide sequence of SEQ ID NO: 1 encodes a DIP2C polypeptide (SEQ ID NO: 2).

Homo sapiens enolase superfamily member 1 (ENOSF1) plays a role in catabolism of L-fucose, a sugar that is part of the carbohydrates attached to cellular glycoproteins. SEQ ID NO: 3, set forth under GenBAnk Accession No. NM_017512.7, is a representative nucleotide sequence encoding ENOSF1. The nucleotide sequence of SEQ ID NO: 3 encodes an ENOSF1 polypeptide (SEQ ID NO: 4).

F-box protein 21 (FBXO21) plays a role in phosphorylation-dependent ubiquitination. SEQ ID NO: 5, set forth under GenBAnk Accession No. NM_033624.3, is a representative nucleotide sequence encoding FBXO21. The nucleotide sequence of SEQ ID NO: 5 encodes a FBXO21 polypeptide (SEQ ID NO: 6).

Homo sapiens potassium channel tetramerization domain containing 6 (KCTD6) is a protein involved in activation of cAMP-dependent Protein Kinase A and the innate immunesystem. SEQ ID NO: 7, set forth under GenBAnk Accession No. NM_153331.3, is a representative nucleotide sequence encoding KCTD6. The nucleotide sequence of SEQ ID NO: 7 encodes a KCTD6 polypeptide (SEQ ID NO: 8).

RNA exonuclease 2 (REXO2) is a 3′-to-5′ exonuclease specific for small (primarily 5 nucleotides or less in length) single-stranded RNA and DNA oligomers. This protein may have a role in DNA repair, replication, and recombination, and in RNA processing and degradation. SEQ ID NO: 9, set forth under GenBAnk Accession No. NM_015523.4, is a representative nucleotide sequence encoding REXO2. The nucleotide sequence of SEQ ID NO: 9 encodes a REXO2 polypeptide (SEQ ID NO: 10).

Homo sapiens major histocompatibility complex, class I, E (HLA-E) is a non-classical MHC class 1 molecule that plays a role in cell recognition by natural killer cells. SEQ ID NO: 11, set forth under GenBAnk Accession No. NM_005516.6, is a representative nucleotide sequence encoding HLA-E. The nucleotide sequence of SEQ ID NO: 11 encodes a HLA-E polypeptide (SEQ ID NO: 12).

Homo sapiens RAB31, member RAS oncogene family (RAB31) is a small GTPases that is a key regulator of intracellular membrane trafficking, from the formation of transport vesicles to their fusion with membrane. SEQ ID NO: 13, set forth under GenBAnk Accession No. NM_006868.4, is a representative nucleotide sequence encoding RAB31. The nucleotide sequence of SEQ ID NO: 13 encodes a RAB31 polypeptide (SEQ ID NO: 14).

Homo sapiens pyridoxal dependent decarboxylase domain containing 1 (PDXDC1) is a protein having carboxy-lyase activity and is associated with pyridoxal phosphate binding. SEQ ID NO: 15, set forth under GenBAnk Accession No. NM_015027.4, is a representative nucleotide sequence encoding PDXDC1. The nucleotide sequence of SEQ ID NO: 15 encodes a PDXDC1 polypeptide (SEQ ID NO: 16).

As used herein, the term “biomarker” includes a polynucleotide or polypeptide molecule which is differentially expressed, i.e., present, increased or decreased in quantity or activity, in subjects having acute rejection or likely to experience acute rejection.

As used herein, the term “gene signature” includes a group of biomarkers, the quantity or activity of each member of which is correlated with subjects having acute rejection or likely to experience acute rejection. In some embodiments, a panel of markers may include only those markers which are either increased in quantity or activity in those subjects. In some embodiments, the panel of markers include one, two, three, four, five, six, seven, or eight, of DIP2C, ENOSF1, FBXO21, KCTD6, PDXDC1, REXO2, HLA-E, and RAB31. In some embodiments, differential expression results in a gene signature wherein DIP2C, ENOSF1, FBXO21, KCTD6, PDXDC1 and REXO2 are downregulated, and wherein HLA-E and RAB31 are upregulated. In some embodiments, the signature is able to distinguish individuals having acute rejection or likely to experience acute rejection from individuals lacking acute rejection or unlikely to experience acute rejection.

As described in the Examples the gene signature provided herein distinguished acute rejection samples from non-acute rejection samples. Classification accuracy was determined using an area under the curve (AUC) measure. The “area under curve” or “AUC” refers to area under a receiver operating characteristic (ROC) curve. AUC under a ROC curve is a measure of accuracy. An area of 1 represents a perfect test, whereas an area of 0.5 represents an insignificant test. A preferred AUC may be between 0.700 and 1. For example, a preferred AUC may be at least approximately 0.700, at least approximately 0.750, at least approximately 0.750, at least approximately 0.800, at least approximately 0.810, at least approximately 0.820, at least approximately 0.830, at least approximately 0.840, at least approximately 0.850, at least approximately 0.860, at least approximately 0.870, at least approximately 0.880, at least approximately 0.890, at least approximately 0.900, at least approximately 0.910, at least approximately 0.920, at least approximately 0.930, at least approximately 0.940, at least approximately 0.950, at least approximately 0.960, at least approximately 0.970, at least approximately 0.980, at least approximately 0.990, or at least approximately 0.995.

As used herein, the “level” of expression means the amount of expression. The level or amount can be described as RNA copy number per microgram of total RNA in a sample or the amount of polypeptide in a sample. As used herein, a “control” or “Baseline level of gene expression” includes the particular gene expression level of a healthy subject or a subject with a well-functioning transplant. The baseline level of gene expression includes the gene expression level of a subject without acute rejection. The baseline level of gene expression can be a reference value, for example, a number on paper, or the baseline level of gene expression from a healthy subject, a subject with a well-functioning transplant, or a subject successfully treated for AR. In some embodiments, the reference value or baseline level of gene expression is from a subject at risk for AR.

In the methods provided herein, the level of expression is determined for one or more of the foregoing genes in a sample obtained from a subject. For example, the quantity of expression of at least two of the foregoing genes, or at least three of the foregoing genes, or at least four of the foregoing genes, or at least five of the foregoing genes, or at least six of the foregoing genes, or at least seven of the foregoing genes, or at least eight of the foregoing genes is determined. In some instances, the quantity of expression of at least five, six, seven, or eight of the foregoing genes is determined. In some embodiments, the quantity of expression of all eight of the foregoing genes is determined.

The term “level of gene expression” as used herein refers to a quantified amount of gene expression. Any procedure available to those of skill in the art can be employed to determine the expression levels of DIP2C, ENOSF1, FBXO21, KCTD6, PDXDC1, REXO2, HLA-E, and RAB31 or a combination thereof. For example, probes, primers, and/or antibodies can be employed in quantitative nucleic acid amplification reactions (e.g., quantitative polymerase chain reaction (PCR)), TAQMAN® assay, primer extension, Northern blot, immunoassay, immunosorbant assay (ELISA), radioimmunoassay (RIA), immunofluorimetry, immunoprecipitation, equilibrium dialysis, immunodiffusion, immunoblotting, mass spectrometry and other techniques available to the skilled artisan. In another example, a microarray can be used. Microarrays are known in the art and consist of a surface to which probes that correspond in sequence to gene products (e.g. mRNAs, rRNAs, polypeptides, fragments thereof etc.) can be specifically hybridized or bound to a known position. Hybridization intensity data detected by the scanner are automatically acquired and processed by the Affymetrix Microarray Suite (MASS) software. Raw data is normalized to expression levels using a target intensity of 150.

A “probe or primer” as used herein refers to a group of nucleic acids where one or more of the nucleic acids can be used to detect one or more genes (e.g., DIP2C, ENOSF1, FBXO21, KCTD6, PDXDC1, REXO2, HLA-E, and RAB31). Exemplary primers and probes for detection of DIP2C, ENOSF1, FBXO21, KCTD6, PDXDC1, REXO2, HLA-E, or RAB31 are set forth in Table 6. It is understood that sequences complementary to the probe sequences set forth in Table 6 are also provided herein. Exemplary primers for amplification of DIP2C, ENOSF1, FBX021, KCTD6, PDXDC1, REXO2, HLA-E, and RAB31 are set forth herein in Table 6. As set forth above, the nucleic acid sequences encoding DIP2C, ENOSF1, FBXO21, KCTD6, PDXDC1, REXO2, HLA-E, and RAB31 are provided herein. Numerous techniques for identifying probes and/or primers, based on these sequences, that specifically amplify any of the biomarkers set forth herein are known to those of skill in the art.

Detection may be, for example, through amplification as in PCR, QPCR, RT-PCR, or primer extension. Detection can also be through hybridization, or through selective destruction and protection, as in assays based on the selective enzymatic degradation of single or double stranded nucleic acids, or by detecting RNA affixed to a solid surface (e.g., a blot). Probes and/or primers may be labeled with one or more fluorescent labels, radioactive labels, fluorescent quenchers, enzymatic labels, or other detectable moieties. Probes may be any size so long as the probe is sufficiently large to selectively detect the desired nucleic acid or to serve as a primer for amplification.

Primers can be polynucleotides or oligonucleotides capable of being extended in a primer extension reaction at their 3′ end. In order for an oligonucleotide to serve as a primer, it typically needs only be sufficiently complementary in sequence to be capable of forming a double-stranded structure with the template, or target, under the conditions employed. Establishing such conditions typically involves selection of solvent and salt concentration, incubation temperatures, incubation times, assay reagents and stabilization factors known to those in the art. The term primer or primer oligonucleotide refers to an oligonucleotide as defined herein, which is capable of acting as a point of initiation of synthesis when employed under conditions in which synthesis of a primer extension product that is complementary to a nucleic acid strand is induced, as, for example, in a DNA replication reaction such as a PCR reaction. Like non-primer oligonucleotides, primer oligonucleotides may be labeled according to any technique known in the art, such as with radioactive atoms, fluorescent labels, enzymatic labels, proteins, haptens, antibodies, sequence tags, mass label or the like. Such labels may be employed by associating them, for example, with the 5′ terminus of a primer by a plurality of techniques known in the art. Such labels may also act as capture moieties. A probe or primer may be in solution, as would be typical for multiplex PCR, or a probe or primer may be adhered to a solid surface, as in an array or microarray. It is well known that compounds such as PNAs may be used instead of nucleic acids to hybridize to genes. In addition, probes may contain rare or unnatural nucleic acids such as inosine.

As used herein, the terms “hybridize” and “hybridization” refer to the annealing of a complementary sequence to the target nucleic acid, i.e., the ability of two polymers of nucleic acid (polynucleotides) containing complementary sequences to anneal through base pairing. The terms “annealed” and “hybridized” are used interchangeably throughout, and are intended to encompass any specific and reproducible interaction between a complementary sequence and a target nucleic acid, including binding of regions having only partial complementarity. Certain bases not commonly found in natural nucleic acids may be included in the nucleic acids of the present invention and include, for example, inosine and 7-deazaguanine. Those skilled in the art of nucleic acid technology can determine duplex stability empirically considering a number of variables including, for example, the length of the complementary sequence, base composition and sequence of the oligonucleotide, ionic strength and incidence of mismatched base pairs. The stability of a nucleic acid duplex is measured by the melting temperature, or “Tm”. The Tm of a particular nucleic acid duplex under specified conditions is the temperature at which on average half of the base pairs have disassociated.

Hybridization reactions can be performed under conditions of different “stringency”. The stringency of a hybridization reaction includes the difficulty with which any two nucleic acid molecules will hybridize to one another. Under stringent conditions, nucleic acid molecules at least 60%, 65%, 70%, 75% identical to each other remain hybridized to each other, whereas molecules with low percent identity cannot remain hybridized. A preferred, non-limiting example of highly stringent hybridization conditions are hybridization in 6×sodium chloride/sodium citrate (SSC) at about 45° C., followed by one or more washes in 0.2×SSC, 0.1% SDS at 50° C., preferably at 55° C., more preferably at 60° C., and even more preferably at 65° C. When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide if hybridization can occur between one of the strands of the first polynucleotide and the second polynucleotide. “Complementarity” or “homology” is quantifiable in terms of the proportion of bases in opposing strands that are expected to hydrogen bond with each other, according to generally accepted base-pairing rules.

The term “stringency” is used in reference to the conditions of temperature, ionic strength, and the presence of other compounds, under which nucleic acid hybridizations are conducted. With “high stringency” conditions, nucleic acid base pairing will occur only between nucleic acid fragments that have a high frequency of complementary base sequences. Thus, conditions of “medium” or “low” stringency are often required when it is desired that nucleic acids which are not completely complementary to one another be hybridized or annealed together. It is well-known in the art that numerous equivalent conditions can be employed to comprise medium or low stringency conditions. The choice of hybridization conditions is generally evident to one skilled in the art and is usually guided by the purpose of the hybridization, the type of hybridization (DNA-DNA or DNA-RNA), and the level of desired relatedness between the sequences (e.g., Sambrook et al. (1989); Nucleic Acid Hybridization, A Practical Approach, IRL Press, Washington D.C. 1985, for a general discussion of the methods).

The stability of nucleic acid duplexes is known to decrease with an increased number of mismatched bases, and further to be decreased to a greater or lesser degree depending on the relative positions of mismatches in the hybrid duplexes. Thus, the stringency of hybridization can be used to maximize or minimize stability of such duplexes. Hybridization stringency can be altered by: adjusting the temperature of hybridization; adjusting the percentage of helix destabilizing agents, such as formamide, in the hybridization mix; and adjusting the temperature and/or salt concentration of the wash solutions. For filter hybridizations, the final stringency of hybridizations often is determined by the salt concentration and/or temperature used for the post-hybridization washes.

“High stringency conditions” when used in reference to nucleic acid hybridization include conditions equivalent to binding or hybridization at 42° C. in a solution consisting of 5.times. SSPE (43.8 g/l NaCl, 6.9 g/l NaH₂PO₄ H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed. In general, the stringency of hybridization is determined by the wash step. Hence, a wash step involving 0.1×SSPE, 1.0% SDS at a temperature of at least 42° C. can yield a high stringency hybridization product. In some instances, the high stringency hybridization conditions include a wash in 1×SSPE, 1.0% SDS at a temperature of at least 50° C., or at about 65° C.

“Medium stringency conditions” when used in reference to nucleic acid hybridization include conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed. Hence, a wash step involving 1.0×SSPE, 1.0% SDS at a temperature of 42° C. can yield a medium stringency hybridization product.

“Low stringency conditions” include conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharmacia), 5 g BSA (Fraction V; Sigma)] and 100 g/ml denatured salmon sperm DNA followed by washing in a solution comprising 5×SSPE, 0.1% SDS at 42° C. when a probe of about 500 nucleotides in length is employed. Hence, a wash step involving 5×SSPE, 1.0% SDS at a temperature of 42° C. can yield low stringency hybridization product.

As used herein, the term polynucleotide or nucleic acid includes nucleotide polymers of any number. The term polynucleotide can, for example, have less than about 200 nucleotides. However, other polynucleotides can have more than 200 nucleotides. Probes and primers are polynucleotides. Primers can, for example, have between 5 and 100 nucleotides, or have about 15 to 100 nucleotides. Probes can have the same or longer lengths. For example, probes can have about 16 nucleotides to about 10,000 nucleotides. The exact length of a particular polynucleotide depends on many factors, which in turn depend on its ultimate function or use. Some factors affecting the length of a polynucleotide are, for example, the sequence of the polynucleotide, the assay conditions in terms of such variables as salt concentrations and temperatures used during the assay, and whether or not the polynucleotide is modified at the 5′ terminus to include additional bases for the purposes of modifying the mass: charge ratio of the polynucleotide, or providing a tag capture sequence which may be used to geographically separate a polynucleotide to a specific hybridization location on a DNA chip, for example.

As used throughout, the term “up-regulation,” “up-regulated,” “increased expression,” “higher expression,” and “higher levels of expression” are used interchangeably herein and refer to the increase or elevation in the amount of a target RNA or polypeptide. “Up-regulation,” “up-regulated,” “increased expression,” “higher expression,” and “increased levels of expression include increases above a baseline (e.g., a control, or reference) level of 10%, 15%, 20%, 25%, 30%, 35%, 40%, 45%, 50%, 55%, 60%, 65%, 70%, 75%, 80%, 85%, 90%, 95%, 100%, 200% or higher.

As used throughout, the term “down-regulation,” “down-regulated,” “decreased expression,” “lower expression,” and “lower levels of expression” are used interchangeably herein and refer to the increase or elevation in the amount of a target RNA or polypeptide. “Down-regulation,” “down-regulated,” “decreased expression,” “lower expression,” and “lower levels of expression” include decreases below a baseline (e.g., a control, or reference) level of 10%, 15%, 20%, 25%, 30%, 35%, 40%, 45%, 50%, 55%, 60%, 65%, 70%, 75%, 80%, 85%, 90%, 95%, 99% or 100%.

In some embodiments, the expression levels DIP2C, ENOSF1, FBXO21, KCTD6, PDXDC1, REXO2, HLA-E, and RAB31 or a combination thereof are determined using respective probes or primers that can hybridize to the DIP2C, ENOSF1, FBX021, KCTD6, PDXDC1, REXO2, HLA-E, and RAB31 genes. Sequences for DIP2C, ENOSF1, FBXO21, KCTD6, PDXDC1, REXO2, HLA-E, and RAB31 are provided herein and are also readily available and can be used to make such probes and primers.

The mRNA for DIP2C, ENOSF1, FBXO21, KCTD6, PDXDC1, REXO2, HLA-E, and RAB31 that are quantified using the methods described herein have RNA sequences that are the same or complementary to those recited above, except that these RNAs have uracil-containing nucleotides instead of the thymine-containing nucleotides recited in the sequences described herein.

The quantities of RNA expression are conveniently expressed as RNA copies per microgram of total RNA. A standard curve of RNA copy numbers in the selected RNA measurement (e.g., PCR) assays can range, for example, from 25 to 5 million copies, 25 to 3 million copies, or from 25 to 2.5 million copies. When mRNA copy numbers are measured as less than 25 can be scored as 12.5 copies per microgram of total RNA.

The DIP2C, ENOSF1, FBXO21, KCTD6, PDXDC1, REXO2, HLA-E, and/or RAB31 quantified using the methods described herein, and the probes and primers described herein can exhibit some variation of sequence from those recited herein. For example, the DIP2C, ENOSF1, FBXO21, KCTD6, PDXDC1, REXO2, HLA-E, and RAB31 quantified using the methods described herein, and the probes and primers described herein, can have at least 60% sequence identity, or at least 65% sequence identity, or at least 70% sequence identity, or at least 80% sequence identity, or at least 90%, or at least 91% sequence identity, or at least 93% sequence identity, or at least 95% sequence identity, or at least 96%, or at least 97% sequence identity, or at least 98% sequence identity, or at least 99% sequence identity, or at least 99.5% sequence identity to the sequences described herein. For example, also provided herein are sequences having at least 60%, 65%, 70%, 754%, 80%, 85%, 90%, 95%, 99% identity to any one of SEQ ID NOs: 1-112.

The term “identity” or “substantial identity”, as used in the context of a polynucleotide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%, 93%, 94%, 95%, 96%, 97%, 98%, or 99%, as compared to a reference sequence using the programs described herein; preferably BLAST using standard parameters, as described below.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, about 20 to 50, about 20 to 100, about 50 to about 200 or about 100 to about 150, in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215:403-410 and Altschul et al. (1977) Nucleic Acids Res. 25:3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10-5, and most preferably less than about 10-20.

An alternative method for determining the level of DIP2C, ENOSF1, FBXO21, KCTD6, PDXDC1, REXO2, HLA-E, and RAB31 or a combination thereof includes the use of molecular beacons and other labeled probes useful in, for example multiplex PCR. In a multiplex PCR assay, the PCR mixture contains primers and probes directed to the DIP2C, ENOSF1, FBXO21, KCTD6, PDXDC1, REXO2, HLA-E, and RAB31 or a combination thereof. Typically, a single fluorophore is used in the assay. The molecular beacon or probe is detected to determine the level of DIP2C, ENOSF1, FBXO21, KCTD6, PDXDC1, REXO2, HLA-E, and RAB31 or a combination thereof. Molecular beacons are described, for example, by Tyagi and Kramer (Nature Biotechnology 14, 303-308, (1996)) and by Andrus and Nichols in U.S. Patent Application Publication No. 20040053284.

Another method includes, for instance, quantifying cDNA (obtained by reverse transcribing the DIP2C, ENOSF1, FBXO21, KCTD6, PDXDC1, REXO2, HLA-E, and RAB31 or a combination thereof using a fluorescence based real-time detection method, such as the ABI PRISM 7500, 7700, or 7900 Sequence Detection System (TaqMan®) commercially available from Applied Biosystems, Foster City, Calif., or similar system as described by Heid et al., (Genome Res. 1996; 6:986-994) and Gibson et al. (Genome Res. 1996; 6:995-1001).

Generally, the level of DIP2C, ENOSF1, FBXO21, KCTD6, PDXDC1, REXO2, HLA-E, and RAB31 or a combination thereof in a sample is upregulated if the quantity of expression of DIP2C, ENOSF1, FBXO21, KCTD6, PDXDC1, REXO2, HLA-E, and RAB31 or a combination thereof is increased beyond a baseline level. In some embodiments, upregulation includes increases above a baseline level of 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000% or higher. In some instances, the “increased expression” means detection of expression that is greater than a baseline level by 2-fold, 3-fold, 5-fold, 7-fold, 8-fold, 9-fold, 10-fold, or more. “Increased expression” can also mean the 8-gene signature score of patients is significantly higher than a healthy individual or another control. The signature score can be calculated using principal component analysis (PCA), where the score of the first principal component will be used as signature score. See, for example, Berglund et al. “Characteristics and Validation Techniques for PCT-Based Gene-Expression Signatures,” Int. J. Genomics, 2017:2354564. As used herein, “significantly higher” means a false discovery rate (FDR)<0.05, nonparametric Mann-Whitney U test.

The level of expression of DIP2C, ENOSF1, FBXO21, KCTD6, PDXDC1, REXO2, HLA-E, and RAB31, or a combination of any thereof, in a sample is down-regulated if the quantity of expression of DIP2C, ENOSF1, FBX021, KCTD6, PDXDC1, REXO2, HLA-E, and RAB31, or a combination of any thereof, is decreased below a baseline. For example, down-regulation can include decreases below a baseline level by 10%, 15%, 20%, 25%, 30%, 35%, 40%, 45%, 50%, 55%, 60%, 65%, 70%, 75%, 80%, 85%, 90%, 95%, 100% or more below the baseline.

The level of expression of DIP2C, ENOSF1, FBXO21, KCTD6, PDXDC1, REXO2, HLA-E, and RAB31, or a combination of any thereof, in a sample is generally unchanged if the quantity of expression of DIP2C, ENOSF1, FBX021, KCTD6, PDXDC1, REXO2, HLA-E, and RAB31, or a combination of any thereof, does not vary significantly from a baseline. In such instances, a sample from a transplanted tissue in patient having unchanged level of measured expression of DIP2C, ENOSF1, FBXO21, KCTD6, PDXDC1, REXO2, HLA-E, and RAB31 or a combination thereof likely is not being rejected (e.g., no acute rejection), and will likely not be rejected. For example, variance from a baseline level of 0.1%, 0.2%, 0.5%, 1%, 2%, 3%, 4% or less is not sufficiently significant and the level of DIP2C, ENOSF1, FBX021, KCTD6, PDXDC1, REXO2, HLA-E, and RAB31 or a combination thereof in a sample is generally unchanged if such variance is measured.

The term “hybridization” includes a reaction in which one or more nucleic acids or polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two single strands forming a duplex structure, three or more strands forming a multi-stranded complex, a single self-hybridizing strand, or any combination of these. A hybridization reaction may constitute a step in a more extensive process, such as the initiation of a PCR reaction, primer extension reaction, or the enzymatic cleavage of a polynucleotide by a ribozyme.

In some embodiments, to accurately assess whether increased or decreased mRNA or rRNA is significant, the measured expression is “normalized” against a selected normalizer. Normalization of gene expression can allow more accurate comparison of levels of expression between samples. In some instances, the method comprises the steps of (a) measuring the gene expression levels of two or more genes selected from the group consisting of DIP2C, ENOSF1, FBXO21, KCTD6, PDXDC1, REXO2, HLA-E, and RAB31 in a biological sample obtained from the subject, (b) normalizing the gene expression levels of the two or more genes to generate normalized gene expression levels, (c) inputting the normalized gene expression values into a classifier that discriminates between a classification for AR or increased likelihood of developing AR and a non-AR classification, wherein the classifier comprises pre-defined weighting values for each of the genes, (d) calculating a probability for having or developing AR based on the normalized gene expression values, to thereby determine if the subject has or is likely to develop AR, and (e) administering an effective amount of a corticosteroid or antibody therapy to the subject if the subject has been determined to have AR or is likely to develop AR.

### Treatment

Any of the methods for determining the likelihood of acute rejection can be performed at any time after an organ transplant. For example, the likelihood of acute rejection can be determined one or more times after about 4 hours, 8 hours, 12 hours, 24 hours, two days, three, days, four days, five days, six days, one week, two weeks, three weeks, one month, two months, three months, four months, five months, five months, six months, seven months, eight months, nine months, ten months, eleven months, one year, 1.5 years, 2 years or more, post-transplantation. In some embodiments, a subject that is likely to develop acute rejection, also has elevated creatinine levels indicate of acute rejection. In some embodiments, a subject that is likely to develop acute rejection, has normal levels of creatinine levels, i.e., a gene signature set forth herein indicates that acute rejection is likely before an increase in creatinine levels.

The methods for determining an increased likelihood of an acute rejection (e.g., acute kidney rejection) can further include informing medical personnel or the patient about the test results. Information about whether the patient will have acute rejection can also be communicated. If the patient is likely to develop a dysfunction (e.g., kidney dysfunction), the patient can be prescribed and/or administered an effective amount of a treatment to delay rejection of the transplanted organ.

The methods of assaying for acute rejection (e.g., kidney rejection) can further include treatment for an acute rejection, such as kidney transplant rejection, acute tubular injury, T cell mediated rejection (TCR) or antibody-mediated rejection (AMR). Such treatment can include administering an increased or decreased dose of an anti-rejection agent already being administered to the subject. In some embodiments, a different anti-rejection agent can be administered. In some embodiments, an anti-rejection agent can be added to the subject's treatment, if the subject is not currently receiving anti-rejection treatment.

Also provided is a method for treating acute rejection (AR) of a transplant in a subject comprising administering an effective amount of corticosteroid or antibody therapy to a subject who has had an organ transplant, wherein two or more genes selected from the group consisting of DIP2C, ENOSF1, FBXO21, KCTD6, PDXDC1, REXO2, HLA-E, and RAB31 exhibit differential expression in a biological sample obtained from the subject as compared to a control.

As used throughout, “effective amount” or “therapeutically effective amount” refers to an amount sufficient to effect beneficial or desirable biological and/or clinical results. The effective amount of any of the therapeutic agents described herein can be determined by one of ordinary skill in the art and includes exemplary dosage amounts for a mammal of from about 0.5 to about 200 mg/kg of body weight of active compound per day, which can be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day. Other factors that influence dosage can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject also depends upon the judgment of the treating medical practitioner. A therapeutically effective amount is also one in which any toxic or detrimental effects of the composition are outweighed by the therapeutically beneficial effects.

As used herein, administer or administration refers to the act of introducing, injecting or otherwise physically delivering a substance as it exists outside the body into a subject, such as by mucosal, intradermal, intravenous, intratumoral, intramuscular, intrathecal, intracranial, intrarectal, oral, subcutaneous delivery and/or any other method of physical delivery described herein or known in the art.

Any of the therapeutic agents described herein are administered in a number of ways depending on whether local or systemic treatment is desired, and on the area to be treated. The compositions are administered via any of several routes of administration, including orally, parenterally, intrathecally, intracranially, intramucosally, intravenously, intraperitoneally, intraventricularly, intramuscularly, subcutaneously, intracavity or transdermally. Administration can be achieved by, e.g., topical administration, local infusion, injection, or by means of an implant. The implant can be of a porous, non-porous, or gelatinous material, including membranes, such as sialastic membranes, or fibers. The implant can be configured for sustained or periodic release of the composition to the subject. See, e.g., U.S. Patent Application Publication No. 20080241223; U.S. Pat. Nos. 5,501,856; 4,863,457; and 3,710,795; and European Patent Nos. EP488401 and EP 430539. In some methods, the therapeutic agent can be delivered to the subject by way of an implantable device based on, e.g., diffusive, erodible, or convective systems, osmotic pumps, biodegradable implants, electrodiffusion systems, electroosmosis systems, vapor pressure pumps, electrolytic pumps, effervescent pumps, piezoelectric pumps, erosion-based systems, or electromechanical systems. Effective doses for any of the administration methods described herein can be extrapolated from dose-response curves derived from in vitro or animal model test systems.

Any of the therapeutic agents described herein can be formulated as a pharmaceutical composition. In some embodiments, the pharmaceutical composition can further comprise a carrier. The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, artificial cerebral spinal fluid, dextrose, and water.

Depending on the intended mode of administration, a pharmaceutical composition comprising a therapeutic agent described herein, can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the agent described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

As used herein the terms “treatment”, “treat”, or “treating” refers to a method of reducing one or more of the effects of the disease or one or more symptoms of the disease, for example, AR, in the subject. Thus, in the disclosed methods, treatment can refer to a 10%, 20%, 30%, 40%, 50%, 60%, 70%, 80%, 90%, or 100% reduction in the severity of AR. In addition to alleviation or prevention of symptoms, treatment can also slow or stop the progression or worsening of a disease, disorder, or condition and/or the remission of the disease, disorder or condition. For example, a method for treating AR is considered to be a treatment if there is a 10% reduction in one or more symptoms of AR in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease or symptoms of the disease.

An “anti-rejection agent” is any substance administered to a subject for the purpose of preventing or ameliorating a rejection state. Anti-rejection agents include, but are not limited to, azathioprine, cyclosporine, FK506, tacrolimus, mycophenolate mofetil, anti-CD25 antibody, antithymocyte globulin, rapamycin, ACE inhibitors, perillyl alcohol, anti-CTLA4 antibody, anti-CD40L antibody, anti-thrombin III, tissue plasminogen activator, antioxidants, anti-CD 154, anti-CD3 antibody, thymoglobin, OKT3, corticosteroid, or a combination thereof.

In some embodiments, the acute rejection relates to acute kidney rejection. It is important to distinguish between acute rejection and drug toxicity. Two of the commonly used drugs prescribed to transplant recipients to prevent rejection, cyclosporine and tacrolimus, can cause kidney toxicity, and this complication is not readily identified solely on the basis of blood concentrations of cyclosporine/tacrolimus. In kidney transplant patients, the clinical importance of distinguishing acute rejection from cyclosporine/tacrolimus toxicity cannot be overemphasized because the treatment approaches are diametrically opposite. In one instance, continued administration of cyclosporine/tacrolimus for rejection is critical whereas, in the other instance, a reduction in dosage or discontinuation of cyclosporine/tacrolimus is indicated to prevent further kidney toxicity. Furthermore, deterioration in kidney function is not always available as a clinical clue to diagnose rejection because many of the kidney transplants suffer from acute (reversible) renal failure in the immediate post-transplantation period due to injury from organ procurement and the ex-vivo preservation procedures involved.

In instances where acute rejection is predicted, a steroid pulse therapy can be started and may include the administration for three to six days of a high dose corticosteroid (e.g., greater than 100 mg or 5-10 mg/kg per kg up to 1 gram). A maintenance regimen of prednisone doses (0.07 to 0.1 mg/kg for children and adults, with a maximum of 5 mg daily) can be used if the patient is not receiving steroid treatment. One or more antibody preparations can be administered for treatment of acute rejection (e.g., acute cellular rejection). Examples of antibody therapy that can be used for treatment of acute rejection include the administration for seven to fourteen days of a lymphocyte-depleting antibody, an anti-thymoglobulin antibody (for example, a polyclonal antibody), an anti-CD52 antibody (for example, alemtuzumab), and an anti-CD3 antibody (e.g., monoclonal antibody OT3). See, for example, Halloran “Immunosuppressive Drugs for Kidney Transplantation,” NEJM 2004; 351:2715-29; and Cooper “Evaluation and Treatment of Acute Rejection in Kidney Allografts,” CJASN 15:430-438 (2020), for additional information regarding treatment of acute rejection.

Another example of a treatment that can be administered, for example for antibody-mediated rejection, is plasmapheresis or plasma exchange. Plasmapheresis is a process in which the fluid part of the blood (i.e., plasma) is removed from blood cells. Typically, the plasma is removed by a device known as a cell separator. The cells are generally returned to the person undergoing treatment, while the plasma, which contains antibodies, is discarded. Other examples, of treatments for antibody mediated acute rejection include intravenous immunoglobulin, and/or anti-CD20 antibodies.

Any of the methods provided herein can further comprise performing a biopsy on transplant tissue from the subject, to determine if antibody-mediated damage has occurred in the subject. The term “biopsy” refers to a specimen obtained by removing tissue from living patients for diagnostic examination. The term includes aspiration biopsies, brush biopsies, chorionic villus biopsies, endoscopic biopsies, excision biopsies, needle biopsies (specimens obtained by removal by aspiration through an appropriate needle or trocar that pierces the skin, or the external surface of an organ, and into the underlying tissue to be examined), open biopsies, punch biopsies (trephine), shave biopsies, sponge biopsies, and wedge biopsies. Biopsies also include a fine needle aspiration biopsy, a minicore needle biopsy, and/or a conventional percutaneous core needle biopsy.

A biopsy can be performed before, concurrently with, or after an assessment of the likelihood of acute rejection of the transplant in the subject. One of skill in the art will appreciate that, in some embodiments, when any of the methods provided herein indicate that acute rejection of the transplant is unlikely, a biopsy is optional, but not necessary until there is a likelihood of acute rejection based on a gene signature described herein and/or an increase in creatinine levels in the subject.

If the biopsy shows antibody-mediated damage, plasma exchange therapy or intravenous immunoglobulin (Ig) therapy can be administered to the subject. In some embodiments, the intravenous Ig therapy is administered in combination with rituximab. It is understood that, if the subject is likely to develop acute rejection, an effective amount of a corticosteroid or antibody (a lymphocyte-depleting antibody, an anti-thymoglobulin antibody (for example, a polyclonal antibody), an anti-CD52 antibody (for example, alemtuzumab), and an anti-CD3 antibody (e.g., monoclonal antibody OT3), as described above, can be administered to the subject prior to obtaining biopsy results. If the biopsy results show antibody-mediated damage, plasma exchange therapy, intravenous Ig therapy, anti-IL6 therapy (for example, tocilizumab), and/or proteosomal inhibitor therapy (for example, bortezomib or carfilzomib) can be administered to the subject, with or without rituximab.

### Kits

Any of the methods provided herein can also be performed by use of kits that are described herein. Provided herein is a kit comprising: (a) agents for detection of two or more genes selected from the group consisting of DIP2C, ENOSF1, FBXO21, KCTD6, PDXDC1, REXO2, HLA-E, and RAB31. In some embodiments, the agents are nucleotide primers or probes. In general, kits can include a detection reagent that is suitable for detecting the presence of one or more RNA of interest. The kits can include a panel of probe and/or primer sets. Such probe and/or primer sets are designed to detect expression of one or more genes and provide information about the rejection of a transplant organ. Probe sets can include probes or primers that are labeled (e.g., fluorescer, quencher, etc.). Unlabeled probes or primers can also be provided in the kits.

The probes and primers are useful for detection of DIP2C, ENOSF1, FBXO21, KCTD6, PDXDC1, REXO2, HLA-E, and RAB31 or a combination thereof. The probe and/or primer sets are targeted at the detection of RNA transcripts and/or structural RNAs that are informative about acute rejection. Probe and/or primer sets may also comprise a large or small number of probes or primers that detect gene transcripts that are not informative about transplant rejection. Such probes and primers are useful as controls and for normalization. Probe and/or primer sets can be provided in the kits as a dry material or dissolved in solution. In some embodiments, probe and/or primer sets can be affixed to a solid substrate to form an array of probes. Probe and/or primer sets can be configured for multiplex PCR. The probes and/or primers can be nucleic acids (e.g., DNA, RNA, chemically modified forms of DNA and RNA), LNA, or PNA, or any other polymeric compound capable of specifically hybridizing with the desired nucleic acid sequences.

The kits can include components for isolating and/or detecting RNA in essentially any sample (e.g., urine, blood, etc.), and a wide variety of reagents and methods are, in view of this specification, known in the art. Hence, the kits can include vials, swabs, needles, syringes, labels, pens, pencils, or combinations thereof.

In some embodiments, commercially available components can also be included in the kits. For example, the kit can include components from QIAGEN, which manufactures a number of components for RNA isolation, including RNEASY, a Total RNA System (involving binding total RNA to a silica-gel-based membrane and spinning the RNA); OLIGOTEX® for isolation of RNA utilizing spherical latex particles; and QIAGEN total RNA kit for In Vitro Transcripts and RNA clean-up.

The kits can include components for fluorescence based real-time detection methods. For example, the kits can include primers for generating cDNA and/or for amplification of mRNA and rRNA. The kits can include components for 5′ nuclease assays employ oligonucleotide probes labeled with at least one fluorescer and at least one quencher. Prior to cleavage of the probe, the fluorescer excites the quencher(s) rather than producing a detectable fluorescence emission. The oligonucleotide probe hybridizes to a target oligonucleotide sequence for amplification in PCR. The nuclease activity of the polymerase used to catalyze the amplification of the primers of the target sequence serves to cleave the probe, thereby causing at least one fluorescer to be spatially separated from the quencher so that the signal from the fluorescer is no longer quenched. A change in fluorescence of the fluorescer and/or a change in fluorescence of the quencher due to the oligonucleotide probe being digested can be used to indicate the amplification of the target oligonucleotide sequence. Although some primers and probes are described in the Examples, other suitable primers and probes can be employed. Probes and primers can be designed using techniques available to those of skill in the art.

The kits can also include any of the following components: materials for obtaining a sample, enzymes, buffers, probes, primers for generating cDNA, primers for amplifying RNA or cDNA, materials for labeling nucleic acids, microarrays, one or more microarray reader, competitor nucleic acids, probes and/or primers for a housekeeping gene for normalization, control nucleic acids, and antibodies.

In some embodiments, the agents for detection of two or more genes selected from the group consisting of DIP2C, ENOSF1, FBXO21, KCTD6, PDXDC1, REXO2, HLA-E, and RAB31, are antibodies. For antibody-based kits, the kit may comprise, for example: (1) a first antibody (e.g., attached to a solid support) which binds a polypeptide corresponding to a biomarker disclosed herein; and, optionally (2) a second, different antibody that binds to either the polypeptide or the first antibody and is conjugated to a detectable label. The antibody-based kits described herein can be used in numerous protein detection methods, for example, immunoassay, immunosorbant assay (ELISA), radioimmunoassay (RIA), immunoblotting, and other techniques available to the skilled artisan.

In further embodiments, kits can include a biological sample collection system. In some embodiments, the biological sample comprises blood. Blood collection systems can include essentially any material useful for obtaining and/or holding a blood sample. Blood collection systems may include, for example, tubing, a beaker, a flask, a vial, a test tube, a container, and/or a lid for a vial, test tube or container (e.g., a plastic container with a snap-on or screw top lid).

In certain embodiments, kits can also include sample test system. A sample test system can include essentially any material that is useful for containing the sample and contacting the sample with the appropriate detection reagents. In some instances, the sample test system can include purification chambers and purification reagents. A sample test system can include, for example, a sample well, which may be part of a multi-well plate, a petri dish, a filter (e.g., paper, nylon, nitrocellulose, PVDF, cellulose, silica, phosphocellulose, or other solid or fibrous surface), a microchannel (which may be part of a microchannel array or a microfluidics device), a small tube such as a thin-walled PCR tube or a 1.5 ml plastic tube, a microarray to which cells or material obtained from the biological sample may be applied, a capillary tube or a flat or curved surface with detection reagent adhered thereto, or a flat or curved surface with material that adheres to proteins or nucleic acids present in the biological sample or in the cells contained in the biological sample.

Kits can include probes that may be affixed to a solid surface to form a customized array. The probes can be any probe that can hybridize to any of the nucleic acids described herein. In some instances, the probes hybridize under medium to high stringency conditions.

Kits may also include a sample preparation system. A sample preparation system comprises, generally, any materials or substances that are useful in preparing the biological sample to be contacted with the detection reagents. For example, a sample preparation system may include materials for separating sample sediments from the fluids, such as centrifuge tube, a microcentrifuge, or a filter (optionally fitted to a tube designed to permit a pressure gradient to be established across the filter). One example of a filter that can be used is a filter within a syringe, such as those available from Zymo Research (see website at zymoresearch.com/columns-plastics/column-filter-assemblies/zrc-gf-filter; e.g., ZRC-GF Filter™). Other components that can be included in the kit include buffers, precipitating agents for precipitating either wanted or unwanted materials, chelators, cell lysis reagents, RNase inhibitors etc.

Collection, presentation and preparation systems can be accomplished in various ways. For example, a filter can be used to separate sample sediments (e.g., cells) from the sample fluids, and the filter may be coated with antibodies suitable for specifically detecting the desired proteins. One of skill in the art would, in view of this specification, readily understand many combinations of components that a kit of the invention may comprise.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### Examples

### An Age-Independent Gene Signature for Monitoring Acute Rejection in Kidney Transplantation

### Human Genomic Data Collection

A total of 1091 renal gene expression profiles were collected from 7 independent NCBI Gene Expression Omnibus datasets: GSE21374, GSE22459, GSE36059, GSE50058, GSE7392, GSE9493, and GSE25902 (pediatric). In addition, 392 gene expression profiles of peripheral blood cells derived from 5 GEO datasets: GSE14346, GSE15296, GSE24223, GSE46474, and GSE20300 (pediatric) were obtained. Complementing the raw expression data, clinical data from a subset of the samples with AR, including both ABMR and TCMR, stable (STA), borderline rejection, chronic allograft nephropathy (CAN), and interstitial fibrosis/tubular atrophy (IF/TA), were also obtained.

### Normalization of Gene Expression Data

Gene expression profiles of all datasets were measured using Affymetrix U133A or U133 Plus 2.0 expression array. Each dataset selected for this study contained clinical outcome data and patients' unique IDs were also collected from series matrix files (GEO) to ensure there was no redundancy in the sample set. Raw Affymetrix expression CEL files from each dataset were robust multi-array average normalized independently using Expression Console Version 1.1 (Affymetrix, Santa Clara, CA). All data were filtered to include those probes on the HG-U133A platform. Batch effects were mitigated using surrogate variable analysis (SVA).

### Selection and Analysis of Institutional Cohort

To further develop a gene signature of early AR, a total of 89 pediatric and adult patients age 1 to 78 transplanted between July 2009 to July 2017 were selected from Duke University's institutional biorepository. They were characterized as AR (39 TCMR, 1 ABMR, and 2 Borderline)—with samples within the 30 days preceding the rejection event—or STA without rejection during the first year after transplantation. Immunosuppression protocols included induction with basiliximab, daclizumab, or rabbit anti-thymocyte globulin, while maintenance regimens included use of tacrolimus, cyclosporine, azathioprine, belatacept, sirolimus, and/or mycophenolate mofetil with or without steroids, including some patients on full steroid withdrawal regimens. Cryopreserved peripheral blood mononuclear cell mRNA expression of genes identified in our microarray data was measured using Applied Biosystems™ TaqMan™ Array Cards and Plates (Thermo Fisher, Waltham, MA). All samples were collected from patients with informed consent and all related procedures were performed with the approval of the Duke Institutional Review Board.

### Statistics Analyses

Mann-Whitney U Tests were used to identify genes that were differentially expressed between AR and STA groups. Multivariable Cox-regression survival analysis for risk of AR (with the multiple variables being different gene expression values) was also used to identify genes associated with freedom from AR. Shotgun Stochastic Search in Regression (SSS) was used for assigning coefficients to genes that were identified in the previous step. Receiver operating characteristic (ROC) curves were used to assess the diagnostic ability of our signatures in a binary classification system. Gene set enrichment analysis was performed using Enrichr. A gene network was created using STRING v11, Reactome pathway analysis and GO Biological Process analysis were also completed. To assess if the expression of selected 8 genes was truly an independent risk factor of AR, a multivariable logistic regression analysis using generalized linear models (glm) including the clinical variables of race, gender, age, and treatment (use of depletional induction, and/or use of belatacept based maintenance immunosuppression), with a p<0.05 considered significant, was used. Statistical analyses were performed using Prism 6 (GraphPad, San Diego, CA), Matlab 2014a (Mathworks, Natick, MA), R 3.4.0 (Project for Statistical Computing Vienna, Austria), STATA 15 (STATA Corp, College Station, TX) or STATISTICA 7 (Dell, Round Rock, Tx).

### Results

### Sample Normalization

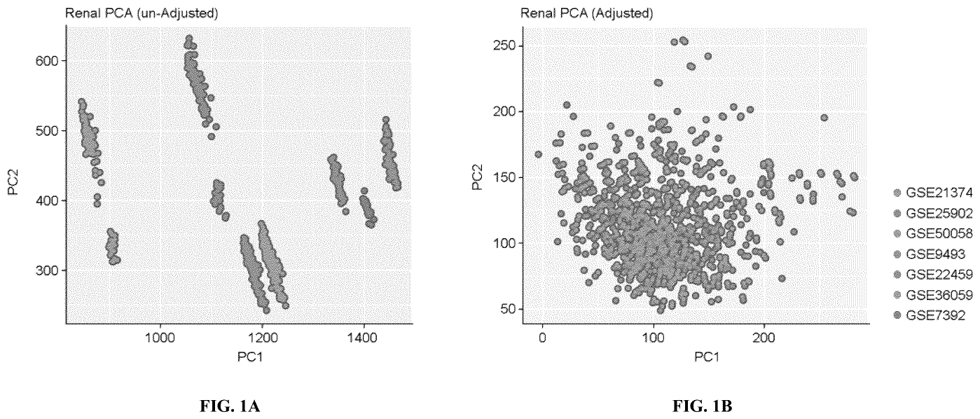

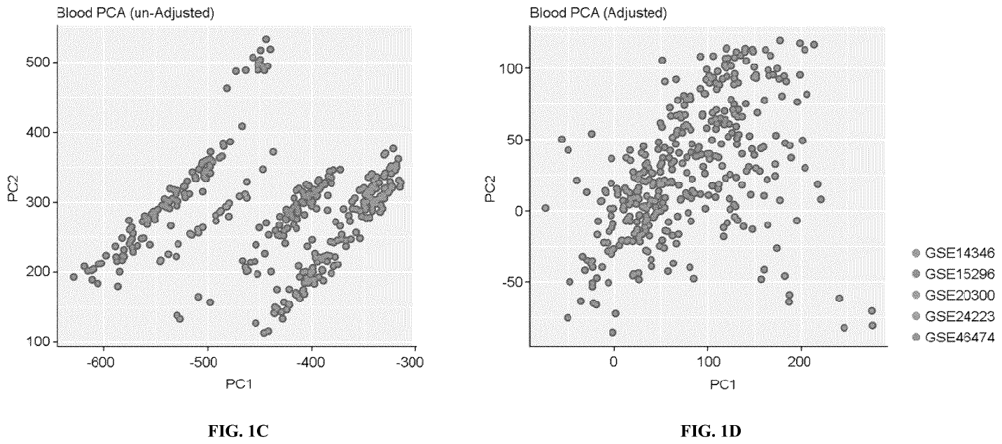

To capture the heterogeneity of renal allograft rejection, we compiled a large collection of gene expression profile data from either kidney allograft parenchymal biopsy specimens (n=1091) or peripheral blood (n=392) obtained from 12 independent public datasets. Allograft and peripheral blood gene expression profiles showed expression differences among samples obtained from different data sets (FIGS. 1A and 1C). All the gene expression data were combined and batch effects in the combined data were corrected using SVA. (FIGS. 1B and 1D).

### Gene Expression Differences Between Adult and Pediatric Samples

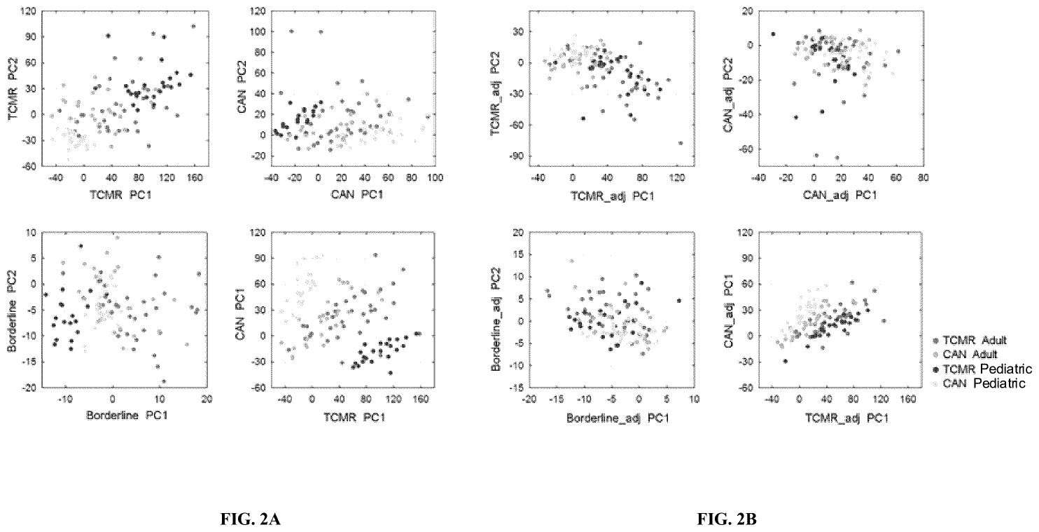

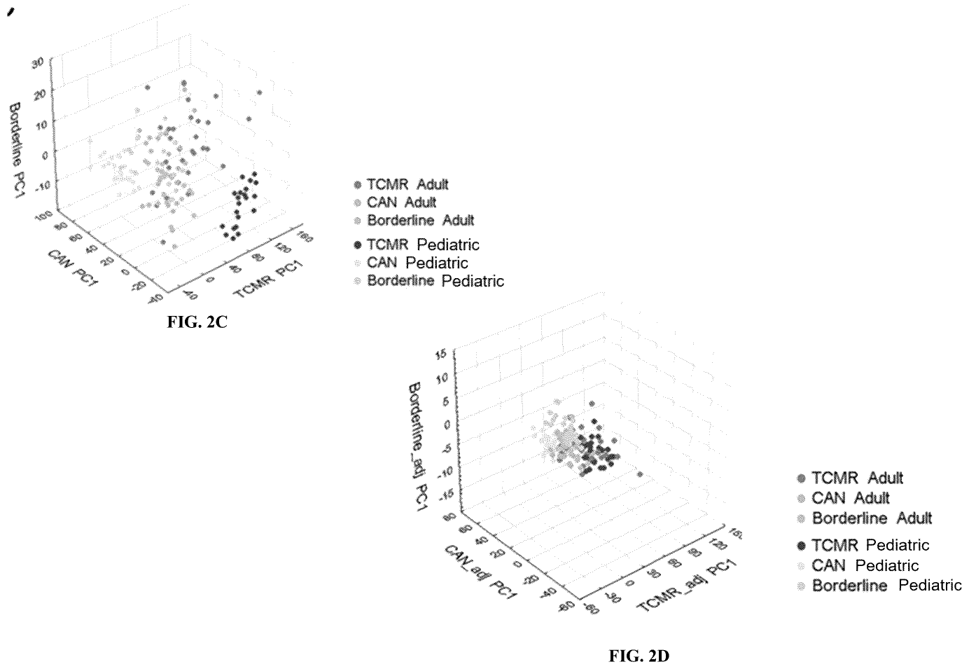

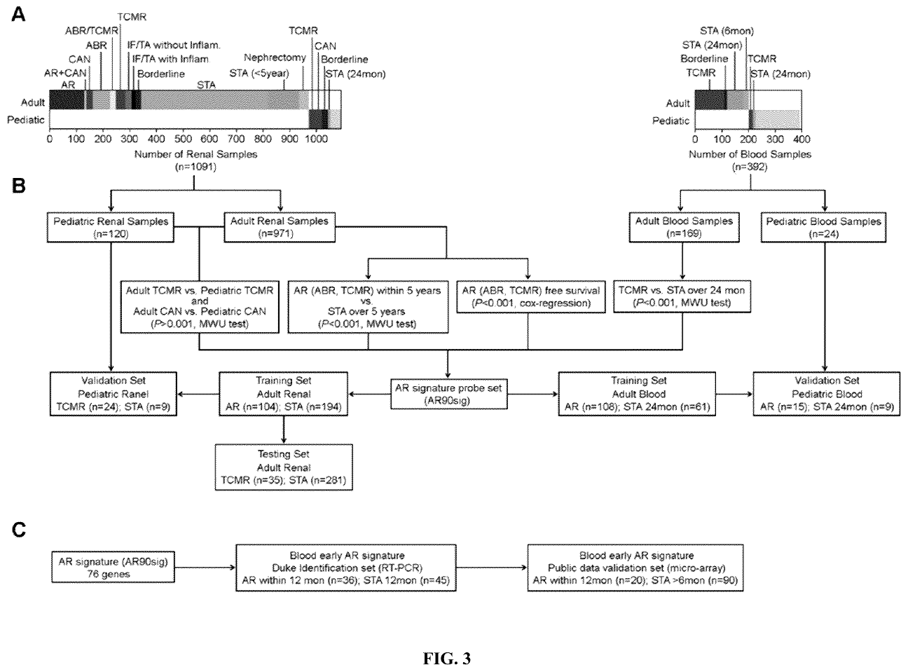

Using all patients (pediatric or adult) 45,782, probe sets were utilized to define expression levels in one of three clinical phenotypes—T Cell Mediated Rejection (TCMR), Borderline rejection, or Chronic Allograft Nephropathy (CAN)—as compared to STA patients (p<0.001, Mann-Whitney U Test). For each of these phenotypes, we plotted both adult and pediatric samples using the first two principal components of differentially expressed probe sets. We observed that adult and pediatric gene expression was significantly different within the TCMR and CAN clinical groups, but not in the borderline group (FIG. 2A and FIG. 2C).

To define the differences between age groups, expression profiles between adult and pediatric samples were subsequently compared and 25,043 probe sets were identified whose expressions in TCMR and/or CAN were significantly different between adult and pediatric samples (p<0.001, Mann-Whitney U Test). After removing these age-group related probe sets, the principal components of TCMR, CAN and borderline were rebuilt using the remaining of 20,739 probe sets. In doing so, a minimization of differences between adult and pediatric samples within the same histologic subtype was observed, indicated by clustering of the points for adult and pediatric samples of the same histologic type (FIGS. 2B and 2D).

### Identification of Gene Expression Differences in AR

To develop an age-independent AR signature, AR associated genes were identified in adult samples using the 20,739 probe sets whose expression was not significantly different between adult and pediatric samples. Allograft gene expression differences were compared between samples with AR within 5 years after kidney transplant to samples without any rejection over five years (STA). Differences in gene expression between adult and pediatric TCMR and CAN were also determined. Genes whose expression patterns were significantly associated with AR-free survival using Cox-regression survival analysis were also identified. Because there was limited long-term follow-up for patients with peripheral blood expression data available in public databases, differences between patients with AR and those that were stable over 2 years were determined. As shown in FIG. 3, these four tests, that were independently performed in either kidney tissue or peripheral blood, identified 90 probe sets whose expression were significantly associated with AR in both allograft and peripheral blood samples by either Mann-Whitney U-Test compared to STA (p<0.001) or by Cox-regression survival analysis (p<0.001) (Table 1). This probe set group (AR90sig) was then utilized to train and test multiple models across sample groups.

TABLE 1
AR associated 90 probe sets

| | | | AR vs. STA | AR vs. STA | AR survival |
|---|---|---|---|---|---|
| Affymetrix | | Expression | (5 year) Renal | (24 mon) Blood | (cox- |
| Probe ID | Symbol | in AR | (MWU test) | (MWU test) | regression) |
| 223660_at | ADORA3 | up | 1.65E−07 | 0.00063 | 9.37E−05 |
| 232304_at | AK026714 | up | 1.82E−08 | 1.56E−07 | 0.00016 |
| 222024_s_at | AKAP13 | up | 7.76E−11 | 0.00011 | 9.16E−08 |
| 227438_at | ALPK1 | up | 2.23E−15 | 2.26E−05 | 2.63E−12 |
| 221039_s_at | ASAP1 | up | 4.30E−12 | 0.00048 | 2.90E−06 |
| 228439_at | BATF2 | up | 9.77E−30 | 0.00030 | 1.97E−18 |
| 242268_at | CELF2 | up | 2.25E−32 | 1.66E−05 | 6.39E−18 |
| 1556323_at | CELF2 | up | 3.89E−12 | 3.31E−07 | 5.86E−12 |
| 211316_x_at | CFLAR | up | 7.70E−12 | 1.13E−07 | 3.57E−06 |
| 211862_x_at | CFLAR | up | 4.39E−05 | 5.47E−05 | 0.00027 |
| 222934_s_at | CLEC4E | up | 3.25E−20 | 2.23E−05 | 2.48E−09 |
| 211287_x_at | CSF2RA | up | 2.71E−25 | 0.00029 | 1.04E−17 |
| 823_at | CX3CL1 | up | 9.54E−16 | 0.00024 | 7.59E−07 |
| 220252_x_at | CXorf21 | up | 1.44E−16 | 4.34E−05 | 1.25E−06 |
| 222858_s_at | DAPP1 | up | 4.55E−23 | 6.74E−05 | 8.36E−09 |
| 1556820_a_at | DLEU2 | up | 1.35E−15 | 1.71E−05 | 9.60E−09 |
| 1556821_x_at | DLEU2 | up | 6.54E−21 | 0.00029 | 3.65E−07 |
| 227780_s_at | ECSCR | up | 1.17E−13 | 6.74E−05 | 3.18E−05 |
| 207610_s_at | EMR2 | up | 6.52E−33 | 0.00026 | 9.67E−10 |
| 214605_x_at | GPR1 | up | 1.28E−15 | 0.00067 | 0.00030 |
| 211040_x_at | GTSE1 | up | 2.67E−18 | 2.04E−05 | 0.00024 |
| 1555629_at | HAVCR2 | up | 4.08E−10 | 0.00021 | 1.45E−05 |
| 230529_at | HECA | up | 1.32E−19 | 3.15E−05 | 8.57E−12 |
| 200905_x_at | HLA-E | up | 2.75E−42 | 9.88E−05 | 3.35E−19 |
| 213418_at | HSPA6 | up | 3.37E−27 | 0.00032 | 2.44E−08 |
| 204786_s_at | IFNAR2 | up | 4.04E−15 | 3.32E−06 | 3.36E−08 |
| 209575_at | IL10RB | up | 1.24E−19 | 0.00074 | 2.08E−13 |
| 210184_at | ITGAX | up | 5.36E−20 | 0.00075 | 4.99E−09 |
| 213803_at | KPNB1 | up | 3.53E−05 | 0.00050 | 0.00015 |
| 207857_at | LILRA2 | up | 7.71E−23 | 0.00072 | 3.36E−07 |
| 232623_at | LOC100128751 | up | 5.21E−12 | 0.00011 | 9.53E−06 |
| 215223_s_at | LOC100129518 | up | 2.28E−10 | 0.00021 | 3.77E−05 |
| 227384_s_at | LOC102725188 | up | 8.33E−25 | 0.00020 | 4.35E−06 |
| 230505_at | LOC145474 | up | 4.22E−09 | 3.78E−08 | 0.00067 |
| 1562511_at | LYST | up | 0.00034 | 1.22E−06 | 4.69E−07 |
| 235421_at | MAP3K8 | up | 4.48E−19 | 2.93E−06 | 2.53E−12 |
| 210869_s_at | MCAM | up | 3.15E−19 | 2.81E−05 | 4.91E−06 |
| 200797_s_at | MCL1 | up | 7.38E−24 | 0.00023 | 4.78E−11 |
| 230805_at | MIR142 | up | 8.83E−15 | 0.00095 | 2.89E−10 |
| 229934_at | mir-223 | up | 1.70E−28 | 0.00035 | 3.48E−12 |
| 208082_x_at | MKRN4P | up | 1.51E−15 | 1.55E−06 | 0.00051 |
| 227066_at | MOB3C | up | 1.60E−07 | 0.00022 | 1.72E−05 |
| 232724_at | MS4A6A | up | 3.53E−32 | 2.69E−07 | 7.96E−12 |
| 214780_s_at | MYO9B | up | 1.66E−12 | 4.93E−06 | 1.85E−05 |
| 1560706_at | NEDD9 | up | 1.09E−24 | 8.17E−08 | 4.93E−08 |
| 201502_s_at | NFKBIA | up | 6.65E−19 | 3.25E−05 | 2.43E−07 |
| 224303_x_at | NIN | up | 2.82E−07 | 0.00028 | 4.50E−05 |
| 228499_at | PFKFB4 | up | 1.27E−10 | 2.80E−08 | 0.00033 |
| 204958_at | PLK3 | up | 2.49E−11 | 0.00034 | 4.01E−07 |
| 38269_at | PRKD2 | up | 2.55E−12 | 0.00092 | 1.74E−06 |
| 217762_s_at | RAB31 | up | 1.04E−26 | 1.83E−06 | 2.78E−11 |
| 232722_at | RNASET2 | up | 4.75E−20 | 6.12E−06 | 5.33E−08 |
| 233880_at | RNF213 | up | 0.00027 | 0.00061 | 1.79E−08 |
| 1566079_at | RPS16P5 | up | 1.26E−13 | 0.00084 | 3.57E−10 |
| 1559882_at | SAMHD1 | up | 4.82E−26 | 0.00051 | 3.01E−13 |
| 211429_s_at | SERPINA1 | up | 0.00030 | 0.00032 | 3.73E−05 |
| 1559034_at | SIRPB2 | up | 5.93E−10 | 9.85E−09 | 6.66E−10 |
| 237426_at | SP100 | up | 6.68E−19 | 3.60E−06 | 1.78E−10 |
| 208392_x at | SP110 | up | 5.01E−22 | 0.00064 | 2.75E−12 |
| 209761_s_at | SP110 | up | 4.99E−20 | 4.96E−05 | 1.01E−11 |
| 1556601_a_at | SPAT13 | up | 1.14E−10 | 0.00029 | 8.40E−06 |
| 1556203_a_at | SRGAP2 | up | 3.99E−11 | 2.36E−05 | 0.00062 |
| 236259_at | STK4 | up | 2.86E−07 | 4.41E−05 | 1.76E−09 |
| 207643_s_at | TNFRSF1A | up | 2.73E−07 | 2.46E−07 | 3.29E−05 |
| 213261_at | TRANK1 | up | 3.01E−26 | 0.00068 | 1.70E−12 |
| 203234_at | UPP1 | up | 5.36E−05 | 0.00015 | 3.43E−05 |
| 211800_s_at | USP4 | up | 3.92E−08 | 2.23E−06 | 1.29E−07 |
| 225273_at | WWC3 | up | 8.85E−12 | 4.63E−06 | 5.64E−07 |
| 212860_at | ZDHHC18 | up | 4.98E−09 | 2.16E−06 | 3.40E−05 |
| 215706_x_at | ZYX | up | 4.44E−09 | 0.00093 | 0.00090 |
| 202053_s_at | ALDH3A2 | down | 1.90E−12 | 5.08E−06 | 9.95E−06 |
| 217988_at | CCNB1IP1 | down | 5.62E−12 | 0.00071 | 5.09E−05 |
| 230656_s_at | CIRH1A | down | 1.38E−11 | 4.04E−07 | 0.00077 |
| 223978_s_at | CRLS1 | down | 4.11E−13 | 1.71E−05 | 1.71E−07 |
| 224870_at | DANCR | down | 5.04E−11 | 2.56E−05 | 2.55E−05 |
| 212503_s_at | DIP2C | down | 2.54E−14 | 0.00012 | 0.00022 |
| 204143_s_at | ENOSF1 | down | 4.27E−12 | 9.34E−07 | 0.00020 |
| 212231_at | FBXO21 | down | 2.00E−10 | 1.78E−08 | 0.00026 |
| 220642_x_at | GPR89A | down | 4.09E−16 | 2.30E−05 | 1.11E−05 |
| 211569_s_at | HADH | down | 6.77E−17 | 8.02E−08 | 0.00041 |
| 201035_s_at | HADH | down | 3.97E−10 | 6.51E−05 | 0.00059 |
| 238001_at | KCTD6 | down | 2.92E−09 | 8.74E−05 | 5.80E−05 |
| 212053_at | LOC102724985 | down | 9.53E−15 | 3.71E−08 | 0.00052 |
| 223177_at | NT5DC1 | down | 1.79E−13 | 3.83E−06 | 0.00092 |
| 203335_at | PHYH | down | 2.92E−15 | 1.04E−05 | 0.00019 |
| 218194_at | REXO2 | down | 1.03E−12 | 0.00014 | 0.00079 |
| 1553118_at | THEM4 | down | 7.54E−12 | 0.00081 | 0.00055 |
| 223105_s_at | TMEM14B | down | 1.18E−05 | 2.58E−05 | 0.00017 |
| 223282_at | TSHZ1 | down | 1.24E−11 | 4.30E−08 | 0.00082 |
| 227907_at | TUBGCP4 | down | 1.23E−09 | 0.00018 | 5.20E−05 |

### Biologic Validity of Candidate Genes

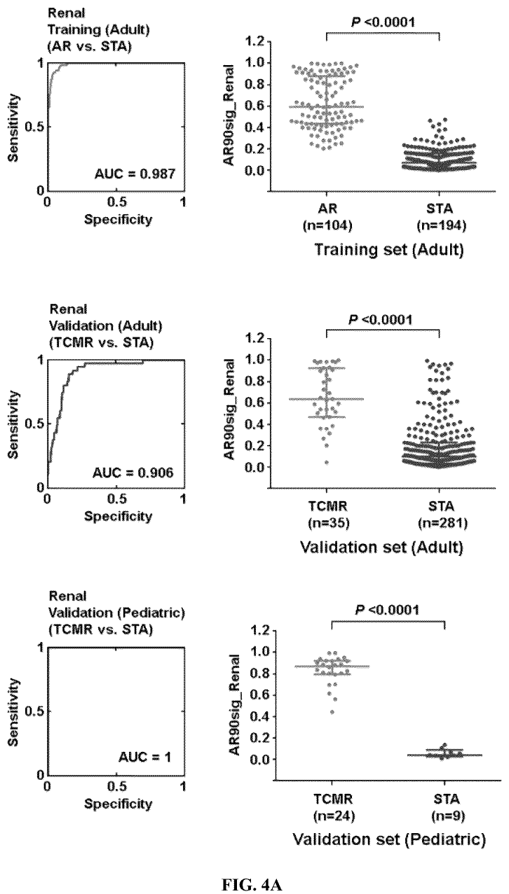

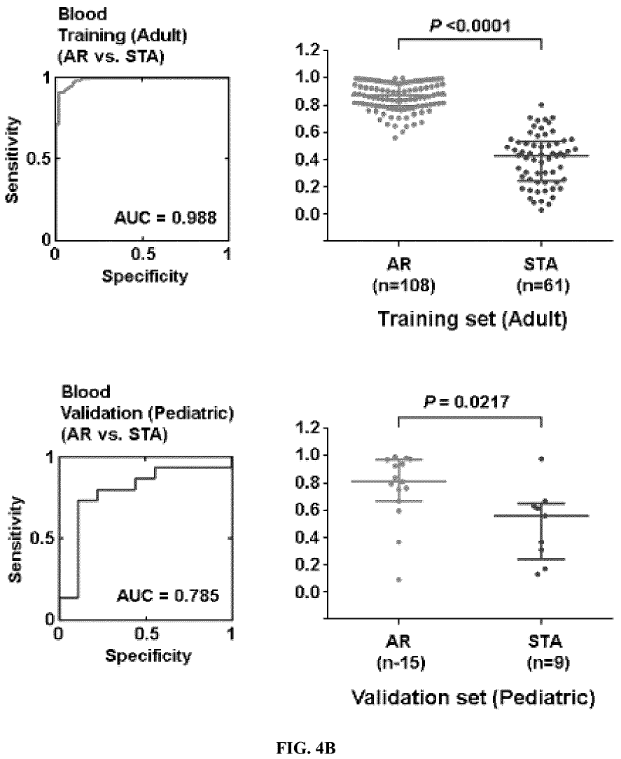

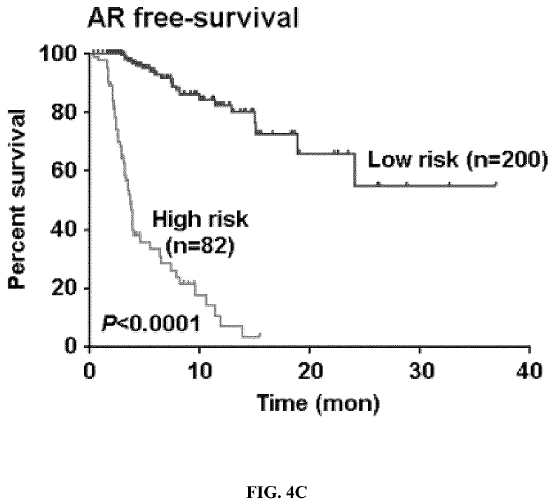

These genes were plotted on a heatmap to define their expression between groups which confirmed good segregation between AR and STA groups (See FIG. 4A, Shaw et al. Theranostics 10 (15): 6977-6986 (2020)). From the 90 AR associated probe sets, we identified 76 genes whose expressions were significantly changed in AR. To determine the biologic basis of these 76 genes, gene set enrichment analysis was performed and the gene signature was found to be significantly associated with immune system and interferon signaling (Table 2; (See FIG. 4A, Shaw et al. Theranostics 10 (15): 6977-6986 (2020)).

TABLE 2
Enrichr gene set enrichment analysis of AR up-regulated gene from AR90

| | Combined | |
|---|---|---|
| | Score | Genes from AR90 |
| Reactome 2016 Terms | | |
| Cytokine Signaling in Immune | 29.21 | IFNAR2, SP100, IL10RB, PELI1, |
| system_Homo sapiens_R-HSA- | | MAP3K8, SAMHD1, CSF2RA, KPNB1, |
| 1280215 | | TNFRSF1A, HAVCR2, HLA-E |
| Immune System_Homo sapiens_R- | 26.21 | IFNAR2, SP100, IL10RB, DAPP1, |
| HSA-168256 | | MYO9B, SAMHD1, LILRA2, CSF2RA, |
| | | TNFRSF1A, HLA-E, NFKBIA, |
| | | RNF213, PELI1, MAP3K8, CLEC4E, |
| | | KPNB1, HAVCR2 |
| Interferon Signaling_Homo | 15.97 | IFNAR2, SP100, SAMHD1, KPNB1, |
| sapiens_R-HSA-913531 | | HLA-E |
| Death Receptor Signalling_Homo | 15.27 | USP4, CFLAR, TNFRSF1A |
| sapiens_R-HSA-73887 | | |
| TNFR1-induced proapoptotic | 12.55 | USP4, TNFRSF1A |
| signaling_Homo sapiens_R-HSA- | | |
| 5357786 | | |
| Interferon alpha/beta | 11.84 | IFNAR2, SAMHD1, HLA-E |
| signaling_Homo sapiens_R-HSA- | | |
| 909733 | | |
| MyD88 cascade initiated on plasma | 11.82 | NFKBIA, PELI1, MAP3K8 |
| membrane_Homo sapiens_R-HSA- | | |
| 975871 | | |
| Toll Like Receptor 10 (TLR10) | 11.78 | NFKBIA, PELI1, MAP3K8 |
| Cascade_Homo sapiens_R-HSA- | | |
| 168142 | | |
| Toll Like Receptor 5 (TLR5) | 11.73 | NFKBIA, PELI1, MAP3K8 |
| Cascade_Homo sapiens_R-HSA- | | |
| 168176 | | |
| TRAF6 mediated induction of | 11.72 | NFKBIA, PELI1, MAP3K8 |
| NFkB and MAP kinases upon | | |
| TLR7/8 or 9 activation_Homo | | |
| sapiens_R-HSA-975138 | | |
| MyD88 dependent cascade initiated | 11.66 | NFKBIA, PELI1, MAP3K8 |
| on endosome_Homo sapiens_R- | | |
| HSA-975155 | | |
| Toll Like Receptor 7/8 (TLR7/8) | 11.62 | NFKBIA, PELI1, MAP3K8 |
| Cascade_Homo sapiens_R-HSA- | | |
| 168181 | | |
| Toll Like Receptor 9 (TLR9) | 11.58 | NFKBIA, PELI1, MAP3K8 |
| Cascade_Homo sapiens_R-HSA- | | |
| 168138 | | |
| MyD88:Mal cascade initiated on | 10.62 | NFKBIA, PELI1, MAP3K8 |
| plasma membrane_Homo | | |
| sapiens_R-HSA-166058 | | |
| Signaling by Interleukins_Homo | 10.60 | IL10RB, PELI1, MAP3K8, CSF2RA, |
| sapiens_R-HSA-449147 | | HAVCR2 |
| Toll Like Receptor 2 (TLR2) | 10.54 | NFKBIA, PELI1, MAP3K8 |
| Cascade_Homo sapiens_R-HSA- | | |
| 181438 | | |
| Toll Like Receptor TLR1:TLR2 | 10.51 | NFKBIA, PELI1, MAP3K8 |
| Cascade_Homo sapiens_R-HSA- | | |
| 168179 | | |
| Toll Like Receptor TLR6:TLR2 | 10.44 | NFKBIA, PELI1, MAP3K8 |
| Cascade_Homo sapiens_R-HSA- | | |
| 168188 | | |
| GO Biological Process 2017 Terms | | |
| type I interferon signaling pathway | 31.72 | IFNAR2, SP100, SAMHD1, HLA-E |
| (GO:0060337) | | |
| neutrophil degranulation | 25.78 | RAB31, SERPINA1, RNASET2, |
| (GO:0043312) | | HSPA6, ITGAX, KPNB1 |
| defense response to protozoan | 22.46 | BATF2, LYST |
| (GO:0042832) | | |
| leukocyte chemotaxis | 22.45 | LYST, CX3CL1 |
| (GO:0030595) | | |
| positive regulation of inflammatory | 20.73 | NFKBIA, CX3CL1, TNFRSF1A |
| response (GO:0050729) | | |
| positive regulation of I-kappaB | 19.82 | AKAP13, PELI1, CFLAR, TNFRSF1A |
| kinase/NF-kappaB signaling | | |
| (GO:0043123) | | |
| apoptotic process (GO:0006915) | 19.17 | NFKBIA, PLK3, CFLAR, STK4 |
| regulation of extrinsic apoptotic | 17.83 | SP100, CFLAR |
| signaling pathway via death domain | | |
| receptors (GO: 1902041) | | |
| positive regulation of interleukin-4 | 17.33 | HAVCR2, HLA-E |
| production (GO:0032753) | | |
| filopodium assembly (GO:0046847) | 16.80 | SPATAI3, SRGAP2 |
| defense response to virus | 16.80 | IFNAR2, SAMHD1, LYST |
| (GO:0051607) | | |
| positive regulation of NF-kappaB | 14.49 | NFKBIA, PRKD2, CFLAR |
| transcription factor activity | | |
| (GO:0051092) | | |
| negative regulation of apoptotic | 14.37 | NFKBIA, PLK3, CFLAR, MCL1 |
| process (GO:0043066) | | |
| negative regulation of DNA binding | 13.28 | NFKBIA, SP100 |
| (GO:0043392) | | |
| regulation of heart contraction | 13.07 | ADORA3, CELF2 |
| (GO:0008016) | | |
| regulation of small GTPase | 12.91 | AKAP13, MYO9B, SRGAP2 |
| mediated signal transduction | | |
| (GO:0051056) | | |
| I-kappaB kinase/NF-kappaB | 10.06 | NFKBIA, TNFRSF1A |
| signaling (GO:0007249) | | |
| KEGG 2019 Human Terms | | |
| TNF signaling pathway | 860.63 | NFKBIA, MAP3K8, CFLAR, CX3CL1, |
| | | TNFRSF1A |
| Legionellosis | 349.55 | NFKBIA, HSPA6 |
| Toxoplasmosis | 292.20 | NFKBIA, IL10RB, HSPA6, TNFRSF1A |
| Toll-like receptor signaling pathway | 248.19 | IFNAR2, NFKBIA, MAP3K8 |
| Amyotrophic lateral sclerosis (ALS) | 234.98 | TNFRSF1A |
| Human cytomegalovirus infection | 227.09 | NFKBIA, AKAP13, IL10RB, CX3CL1, |
| | | TNFRSF1A, HLA-E |
| Osteoclast differentiation | 208.76 | IFNAR2, NFKBIA, LILRA2, |
| | | TNFRSF1A |
| NF-kappa B signaling pathway | 174.63 | NFKBIA, CFLAR, TNFRSF1A |
| Graft-versus-host disease | 124.12 | HLA-E |
| Autoimmune thyroid disease | 122.23 | HLA-E |
| Complement and coagulation | 118.73 | SERPINA1, ITGAX |
| cascades | | |
| RIG-I-like receptor signaling | 104.87 | NFKBIA |
| pathway | | |
| Necroptosis | 101.20 | IFNAR2, CFLAR, TNFRSF1A |
| Allograft rejection | 99.26 | HLA-E |
| Fructose and mannose metabolism | 79.48 | PFKFB4 |
| Chagas disease (American | 74.84 | NFKBIA, CFLAR, TNFRSF1A |
| trypanosomiasis) | | |
| Cytosolic DNA-sensing pathway | 71.03 | NFKBIA |
| C-type lectin receptor signaling | 67.63 | NFKBIA, PLK3, CLEC4E |
| pathway | | |
| Natural killer cell mediated | 64.90 | IFNAR2, HLA-E |
| cytotoxicity | | |
| Antigen processing and presentation | 64.16 | HSPA6, HLA-E |
| Sphingolipid signaling pathway | 62.77 | ADORA3, TNFRSF1A |
| Shigellosis | 62.35 | NFKBIA |
| Small cell lung cancer | 61.00 | NFKBIA |
| T cell receptor signaling pathway | 49.01 | NFKBIA, MAP3K8 |
| Adipocytokine signaling pathway | 47.81 | NFKBIA, TNFRSF1A |
| B cell receptor signaling pathway | 45.05 | NFKBIA, DAPP1 |
| Chronic myeloid leukemia | 41.08 | NFKBIA |
| Viral myocarditis | 32.66 | HLA-E |
| Apoptosis | 32.28 | NFKBIA, CFLAR, TNFRSF1A, MCL1 |
| Pyrimidine metabolism | 31.50 | UPP1 |
| Th17 cell differentiation | 31.48 | NFKBIA |
| Type I diabetes mellitus | 30.04 | HLA-E |
| Measles | 29.58 | IFNAR2, NFKBIA, HSPA6 |
| Tuberculosis | 27.24 | PLK3, IL10RB, ITGAX, CLEC4E, |
| | | TNFRSF1A |
| IL-17 signaling pathway | 22.49 | NFKBIA |
| Th1 and Th2 cell differentiation | 19.51 | NFKBIA |
| Non-small cell lung cancer | 17.33 | STK4 |
| Insulin resistance | 15.55 | NFKBIA, TNFRSF1A |
| Influenza A | 15.48 | IFNAR2, NFKBIA, HSPA6, |
| | | TNFRSF1A |
| Fc gamma R-mediated phagocytosis | 13.31 | ASAP1 |
| Longevity regulating pathway | 13.28 | HSPA6, SOD2 |
| JAK-STAT signaling pathway | 11.25 | IFNAR2, IL10RB, CSF2RA, MCL1 |
| Parathyroid hormone synthesis, | 11.15 | AKAP13 |
| secretion and action | | |
| Human immunodeficiency virus 1 | 11.07 | NFKBIA, SAMHD1, TNFRSF1A, HLA-E |
| infection | | |
| Hepatitis C | 10.88 | IFNAR2, NFKBIA, CFLAR, |
| | | TNFRSF1A |
| Peroxisome | 10.60 | SOD2 |
| FoxO signaling pathway | 10.44 | PLK3, STK4, SOD2 |
| Leishmaniasis | 10.38 | NFKBIA |

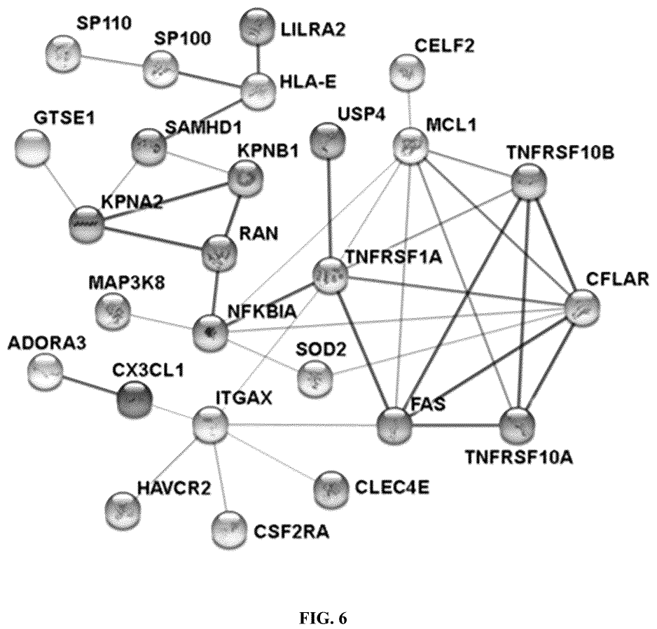

Furthermore, a novel gene network using STRING v11, that included the pathways noted above as well as others (FIG. 6), was defined.

### Developing a 90-Probe Set Identifier of Acute Rejection

Using SSS modeling, a 90-probe set predictor, using a training set of 298 adult kidney allograft samples, was created and validated in independent sets of adult (n=316) and pediatric (n=33) samples (FIG. 4A).

All three analyses showed high sensitivity and specificity for the signature to identify AR. Because the kidney tissue and peripheral blood samples were normalized differently (as they are from different tissue compartments with different background variability), renal tissue and peripheral blood models were built and validated independently. Therefore, a separate signature in adult peripheral blood (n=196) samples was created and validated using an independent set of pediatric peripheral blood (n=24) samples (FIG. 4B). Though the two signatures contained the same genes, SSS was run on allograft and peripheral blood samples independently, yielding different coefficients. Of note, the 90-probe set signature performed well on ROC analysis with a minimum area under the curve (AUC) of 0.79 when considering both analyses.

Furthermore, a cut-off gene expression level at maximum sensitivity and specificity in training data to define high vs. low risk of AR was created and this cut-off was applied to validation sets. The positive predictive value (PPV) in the adult renal validation set was 30%, while the negative predictive value (NPV) was at 98%. The model also successfully delineated AR event-free survival between high vs. low risk cases (p<0.0001, Mantel-Cox test) (FIG. 4C). In peripheral blood, a similar analysis was performed which showed a PPV of 85% and NPV of 70% in the pediatric validation dataset (Table 3).

TABLE 3
ROC and likelihood ratios

| | | ROC | Positive Predictive | | Negative Predictive | |
|---|---|---|---|---|---|---|
| | | (AUC) | Value PPV (95% CI) | | Value NPV (95% CI) | |
| Renal AR90 | Adult Training | 0.99 | 94% | (87.67%-97.19%) | 94.95% | (91.25%-97.14%) |
| predictor | Adult Validation | 0.91 | 31.35% | (31.22%-43.92%) | 98.28% | (95.78%-99.31%) |
| | Pediatric | 1 | 100% | | 100% | |
| | Validation | | | | | |
| Blood AR90 | Adult Training | 0.99 | 94.59% | (89.11%-97.40%) | 94.83% | (85.69%-98.25%) |
| predictor | Pediatric | 0.79 | 85.71% | (63.26%-95.43%) | 70% | (44.44%-87.19%) |
| | Validation | | | | | |
| Early AR8 | Duke dataset | 0.71 | 70.59% | (57%-81.29%) | 74.47% | (64.17%-82.61%) |
| predictor | Public dataset | 0.77 | 66.67% | (16%-95.45%) | 83.18% | (81.01-85.14%) |

### Creating an Age-Independent 8 Gene Signature of Early Onset AR

In order to monitor early AR events, blood samples from AR (n=42) and STA (n=47) patients, available from our institutional biorepository, were obtained. All samples were from patients monitored for the first year post transplant, with STA defined as no rejection during that time. All AR samples were obtained within 30 days prior to an AR event. Patients were excluded from the AR group if they experienced another event (e.g. an infection) up to 14 days after the rejection event. Patients in the two groups were demographically similar except with regards to immunosuppressive management. More patients in the AR group received basiliximab induction and/or belatacept maintenance, while patients in STA group received tacrolimus (Table 4).

TABLE 4
Demographics of institutional cohort

| | Rejection- | Stable- | |
|---|---|---|---|
| Characteristic-n (%) | 42 (39) | 47 (44) | P-Value |

| | | | | | |
|---|---|---|---|---|---|
| Age-mean(SD) | 41 | (17) | 39 | (21) | 0.63 |
| Pediatric-n(%) | 8 | (19) | 15 | (32) | 0.23 |
| Female Sex-n(%) | 17 | (41) | 20 | (43) | 1.0 |
| Race-n(%) | | | | | 0.054 |
| African American | 24 | (57) | 13 | (28) | |
| Asian | 0 | (0) | 2 | (4) | |
| White | 16 | (38) | 30 | (64) | |
| Other | 2 | (5) | 2 | (4) | |
| Transplant Type-n(%) | | | | | 0.162 |
| Living Donor | 13 | (38) | 8 | (25) | |
| Deceased donor | 21 | (62) | 24 | (75) | |
| Induction Type-n(%) | | | | | |
| Basiliximab | 14 | (33) | 7 | (15) | 0.049 |
| Anti-Thymocyte Globulin | 9 | (21) | 9 | (19) | 0.79 |
| No Induction | 19 | (46) | 31 | (66) | 0.058 |
| Maintenance Therapy-n(%) | | | | | |
| Prednisone | 40 | (95) | 39 | (82) | 0.095 |
| Tacrolimus | 35 | (83) | 46 | (98) | 0.024 |
| Mycophenolate Mofetil | 42 | (100) | 46 | (98) | 1.0 |
| Cyclosporine | 0 | (0) | 1 | (2) | 1.0 |
| Azathioprine | 1 | (2) | 2 | (4) | 1.0 |
| Sirolimus | 2 | (5) | 4 | (9) | 0.67 |
| Belatacept | 7 | (17) | 0 | (0) | 0.004 |

After removing genes located on the X chromosome, as there are differential numbers between women and men, and probe sets related to microRNA, a total of 76 genes corresponding to the original 90 probe set were interrogated by Real Time-Polymerase Chain Reaction (RT-PCR). 8 genes (DIP2C, ENOSF1, FBXO21, KCTD6, PDXDC1, REXO2, HLA-E, and RAB31) were found to be differentially expressed in AR and this signature retained its significance after adjusting for multiple clinical variables, including as race, gender, age, and treatment (use of depletional induction, and/or use of belatacept based maintenance immunosuppression) (Table 5).

TABLE 5
Early AR (1 year) associated 8 genes

| | Unadjusted P- | Adjusted* P- | Adjusted* OR |
|---|---|---|---|
| Gene | Vale | Value | (2.5%-97.5% CI) |
| HLA-E | 0.01255 | 0.03680 | 5.09 (1.24-26.56) |
| RAB31 | 0.00557 | 0.00641 | 3.83 (1.58-11.21) |
| REXO2 | 0.02985 | 0.02090 | 0.51 (0.28-0.87) |
| ENOSF1 | 0.00150 | 0.00064 | 0.50 (0.32-0.72) |
| DIP2C | 0.01278 | 0.00253 | 0.50 (0.30-0.75) |
| KCTD6 | 0.00093 | 0.00192 | 0.40 (0.21-0.68) |
| PDXDC1 | 0.00117 | 0.00079 | 0.37 (0.19-0.63) |
| FBXO21 | 0.00392 | 0.00089 | 0.25 (0.10-0.53) |

*Adjusted values based on multivariable generalized linear model including age, race, gender, use of depletional induction immunosuppression, and use of belatacept based maintenance immunosuppression

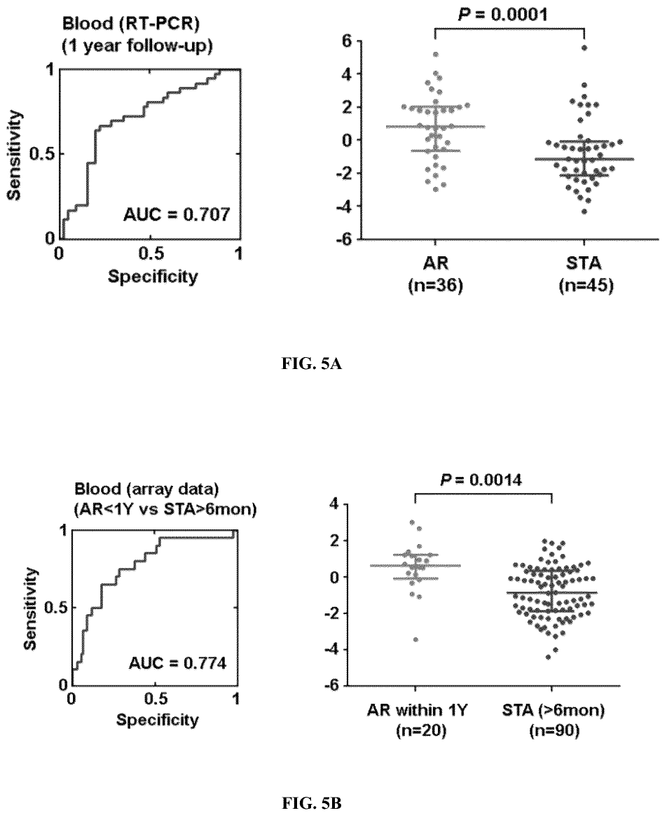

Using these 8 genes, PCA was again employed to create a model to identify AR. The ROC curve AUC was found to be 0.71 (FIG. 5A).

Finally, this signature of early AR events that was validated in 110 Duke patients (adult and pediatric) was applied to patients in the public dataset using the microarray data in the GEO that was initially queried. Utilizing the PCA created with the institutional cohort, the 8 gene signature was applied. This yielded an AUC of 0.77 in this cohort (FIG. 5B). The NPV and PPV for the institutional dataset were 74.5% and 70.6% respectively. The NPV and PPV for the validation in the public dataset were 83.2% and 66.7% respectively.

In the present study, a gene signature for AR, using both publicly available kidney allograft parenchymal and peripheral blood gene expression data and peripheral blood biospecimens from our institutional biorepository, was created and validated. After creation of a 90-probe-set signature targeting 76 genes based on microarray data, validation of this allograft biopsy signature showed a very high AUC in adult (0.91) and pediatric (1.00) datasets. In peripheral blood, validation AUC in a pediatric cohort was moderate at 0.79. Examination of this institutional cohort identified a subset of 8 differentially expressed genes. This 8 gene signature was confirmed in a cohort of 110 patients from public databases and again demonstrated a reasonable AUC for identifying early acute rejection (0.77). Overall, this analysis demonstrates an effective method for biomarker discovery utilizing a combination of publicly available data and single center resources. Provided herein is an age-independent signature of AR that performs well in a peripheral blood assay despite diverse and non-standardized immunosuppressive regimens.

Though there is considerable excitement regarding the ability of peripheral blood-based biomarkers to advance the diagnosis and treatment of disease, there have been great challenges in moving from the research setting into clinical care. Additionally, all biomarker research has been plagued by a lack of reproducibility.

Given these limitations, novel methods of merging available data in all relevant combinations to imbue richness in analysis is needed. As shown herein, building a base set of differentially expressed genes from both kidney allograft and peripheral blood gene expression data, allowed for the detection of a very broad set of relevant genes involved in the AR response. Prior studies have failed to find a strict correlation between genes active in the graft and peripheral blood at the time of AR. The studies described herein, however, show that it can be effective to utilize genes differentially expressed in either compartment in the determination of molecular perturbations in both.

Mechanistically, the 90-probe set signature contained 76 genes, many of which are important in immune regulation. One central pathway in the signature is that of Tumor Necrosis Factor-α (TNF-α) and the nuclear factor K-light-chain-enhancer of activated B-Cells (NFKB) signaling. This multifaceted pathway is important in pro-inflammatory and apoptotic mechanisms depending on the context. The signature of AR was also associated with inflammatory TNF signaling as MCL1, a known anti-apoptotic factor important in both polymorphonuclear cell and lymphocyte survival. Additionally, there was upregulation of USP4 and NFKBIA, both of which downregulate TNF-α based NFKB signaling. These mediators may attenuate overall TNF-α signaling to prevent exhaustion of activated cells. Additionally, some reports in transplantation have noted certain polymorphisms of NFKBI are associated with AR, suggesting that some forms of this gene product may enhance pro-inflammatory signaling. NFKBI is necessary for TNF signaling as it holds NFKB in the cytoplasm prior to nuclear translocation and activation of its inflammatory transcriptional program.

Consistent with an initial analysis, upregulation of Human Leukocyte Antigen (HLA)-E, in the subset of 8 genes that were associated with AR arising within 1-year post-transplant, was observed. HLA-E interacts with CD159c/NKG2C, which activates NK cells. This HLA-E mediated signaling has been shown to occur in the kidney during AR. Interestingly, HLA-E upregulation has been noted as a “Universal” rejection feature of AR, regardless of histologic type. Additionally, two other Class-I HLA presentation associated transcripts, KCTD6 and FBXO21, are implicated in the gene signature. Both are involved in ubiquitination and antigen processing, suggesting a contribution of increased antigen presentation as a contributing factor to rejection.

As shown herein, an age-independent peripheral signature of acute rejection was identified that is effective in the setting of diverse, non-standardized immunosuppressive therapies. This signature provides a less invasive method of identifying acute rejection, to maximize graft survival and minimize patient morbidity.

TABLE 6
Probe Sequences

| | SEQ ID | | |
|---|---|---|---|
| Context Sequence | NO: | Gene Symbol | Gene Name |
| CCATTGGAGGGCAAGTCTGGTGCCA | 17 | 18S | Eukaryotic 18S rRNA |
| GGAACACCTTCCCGCTGCTGGCTCC | 18 | ADGRE2 | adhesion G protein-coupled receptor E2 |
| GTTCAAGACGGCTAAGTCCTTGTTT | 19 | ADORA3 | adenosine A3 receptor |
| TGAAAGAGGTTCAAGCAGTTCTTCT | 20 | AKAP13 | A-kinase anchoring protein 13 |
| CTCAGTATTTAGACCAGGATCTCTA | 21 | ALDH3A2 | aldehyde dehydrogenase 3 family |
| | | | member A2 |
| CGGATAAAAAGGGCCTCTCCACGTC | 22 | ALPK1 | alpha kinase 1 |
| GTGGTGGATTGGCCACATCGAAGGA | 23 | ASAP1 | ArfGAP with SH3 domain, ankyrin repeat |
| | | | and PH domain 1 |
| CTGCACCAGCAGCACGAGTCTCTGG | 24 | BATF2 | basic leucine zipper ATF-like |
| | | | transcription factor 2 |
| CAATCTCCAGAGCCATCGCCTGTGA | 25 | BAZ1A | bromodomain adjacent to zinc finger |
| | | | domain 1A |
| TGGACATATCAGGTACATCAGGAAC | 26 | CCNB1IP1 | cyclin B1 interacting protein 1 |
| TCTGGCGCTCCTGCAGCAGGCCACC | 27 | CELF2 | CUGBP, Elav-like family member 2 |
| CAAGATAAGCAAGGAGAAGAGTTTC | 28 | CFLAR | CASP8 and FADD like apoptosis |
| | | | regulator |
| GCAACAGGCAAGAACGGAGATACTC | 29 | Cirh1a | cirrhosis, autosomal recessive 1A |
| | | | (human) |
| CAGTATGCTGTCTTGCCGACGGGGC | 30 | CKLF | chemokine-like factor |
| GACAAAGTCTCTGAGCTTCTGGGAT | 31 | CLEC4E | C-type lectin domain family 4 member E |
| ACAGATTTGTTGGATGGATTTATTG | 32 | CRLS1 | cardiolipin synthase 1 |
| GCTGGCTGGACAGCACCACGGTGTG | 33 | CX3CL1 | C-X3-C motif chemokine ligand 1 |
| GAGTTGCGCGGGCTGACGCGCCACT | 34 | DANCR | differentiation antagonizing non-protein |
| | | | coding RNA |
| CTGGTCAAGACCTGGAAAACAAGAT | 35 | DAPP1 | dual adaptor of phosphotyrosine and 3- |
| | | | phosphoinositides 1 |
| AGAACACCTTTGAGGTGTTTCCCAT | 36 | DIP2C | disco interacting protein 2 homolog C |
| TAAGAGGATATGAAAGGTGTAAATT | 37 | DLEU2 | deleted in lymphocytic leukemia 2 (non- |
| | | | protein coding) |
| TGGTACCCCAGGCGCAGGTGTCCCC | 38 | ECSCR | endothelial cell surface expressed |
| | | | chemotaxis and apoptosis regulator |
| GCTGCGGCTGCAGAAGGCCAGGCCC | 39 | EHBP1L1 | EH domain binding protein 1 like 1 |
| TACTCACACACCCTATTTTCAAGGC | 40 | ENOSF1 | enolase superfamily member 1 |
| CGTATCTTGAAGGTGCTGTATATAT | 41 | FBXO21 | F-box protein 21 |
| TCACGCACTCTGATGGTGCTGGAAA | 42 | FYB | FYN binding protein |
| CGCTGCCAAGGCTGTGGGCAAGGTC | 43 | GAPDH | glyceraldehyde-3-phosphate |
| | | | dehydrogenase |
| AGTCCACACAGGCTGGAGACGACCT | 44 | GAS7 | growth arrest specific 7 |
| CAACCACTTCAGCTTGACCCTCAAC | 45 | GPI | glucose-6-phosphate isomerase |
| AGTAAGAAGTTCCAAGCTCGCTTCC | 46 | GPR1 | G protein-coupled receptor 1 |
| GGGCTGCGGAGAAGCCCAAGAAAGA | 47 | GTSE1 | G2 and S-phase expressed 1 |
| GTTTGCTGCTGAACATACAATCTTT | 48 | HADH | hydroxyacyl-CoA dehydrogenase |
| CTACTTACAAGGTCCTCAGAAGTGG | 49 | HAVCR2 | hepatitis A virus cellular receptor 2 |
| ACCTTCAAGGGAGACTCATGCATCT | 50 | HECA | hdc homolog, cell cycle regulator |
| GCTTCACCTGGAGCCCCCAAAGACA | 51 | HLA-E | major histocompatibility complex, class I, |
| | | | E |
| TGGTCAAGGTCGCAAGCTTGCTGGT | 52 | HPRT1 | hypoxanthine phosphoribosyltransferase 1 |
| CCGCGCCACCCGGCTGAGTCAGCCC | 53 | HSPA6 | heat shock protein family A (Hsp70) |
| | | | member 6 |
| TTCATATGATTCGCCTGATTACACA | 54 | IFNAR2 | interferon alpha and beta receptor subunit |
| | | | 2 |
| AACAACCCATGACGAAACGGTCCCC | 55 | IL10RB | interleukin 10 receptor subunit beta |
| TGTCCAGGCAGGAGTGCCCAAGACA | 56 | ITGAX | integrin subunit alpha X |
| ATCTACCCTTAACAGAGCTTTTCTT | 57 | KCTD6 | potassium channel tetramerization |
| | | | domain containing 6 |
| GGTGGAAGTGTTGGGTGGTGAATTC | 58 | KPNB1 | karyopherin subunit beta 1 |
| TGGTGGTGACAGGAGCCTACAGCAA | 59 | LILRA2 | leukocyte immunoglobulin like receptor |
| | | | A2 |
| TGCTGGCTACAACAGATTTGTTCTG | 60 | LILRB1 | leukocyte immunoglobulin like receptor |
| | | | B1 |
| AGGGCCGCCCTCCACACCTGGTCTG | 61 | LILRB3; LILRA6 | leukocyte immunoglobulin like receptor |
| | | | B3, leukocyte immunoglobulin like |
| | | | receptor A6 |
| TTCTCTTCAACGGAGCTAAGGTTGG | 62 | LYST | lysosomal trafficking regulator |
| CCTCCTACCTGTACATAATCCACAA | 63 | MAP3K8 | mitogen-activated protein kinase kinase |
| | | | kinase 8 |
| GGAGAAGAACCGGGTCCACATTCAG | 64 | MCAM | melanoma cell adhesion molecule |
| TAAACAAAGAGGCTGGGATGGGTTT | 65 | MCL1 | BCL2 family apoptosis regulator |
| TAACAGCACTGGAGGGTGTAGTGTT | 66 | MIR142 | microRNA 142 |
| TTTCCCACGCGTGTTGGAGTTCCCT | 67 | MOB3C | MOB kinase activator 3C |
| AACCAAGCTTTTGGTGCATAGCAGC | 68 | MS4A6A | membrane spanning 4-domains A6A |
| GAGGAGAGCAACTTCCCCCACGCCA | 69 | MYO9B | myosin IXB |
| GAACATCATCAGCTGAGCCAGTTCC | 70 | NEDD9 | neural precursor cell expressed, |
| | | | developmentally down-regulated 9 |
| TGTCAATGCTCAGGAGCCCTGTAAT | 71 | NFKBIA | NFKB inhibitor alpha |
| GGCTTCTCAGGAAAAGGTTCAGAAT | 72 | NIN | ninein |
| TTTCCGGACACTCGAGAATGATGAG | 73 | NT5DC1 | 5′-nucleotidase domain containing 1 |
| CTACAACGTGGATCAGCACCGGAAG | 74 | ODF3B | outer dense fiber of sperm tails 3B |
| TTGGAGATATATAAGGCTTTCAGTT | 75 | PARP9 | poly(ADP-ribose) polymerase family |
| | | | member 9 |
| CCAGGCTCAGATCCGGTGTTTAAAG | 76 | PDXDC1 | pyridoxal dependent decarboxylase |
| | | | domain containing 1 |
| GAGGAAAAATGGTGGAAATTGAAAC | 77 | PELI1 | pellino E3 ubiquitin protein ligase 1 |
| GGGGGACATGTGGCGGTTTTTGATG | 78 | PFKFB4 | 6-phosphofructo-2-kinase/fructose-2,6- |
| | | | biphosphatase 4 |
| AGCCCACAGCTCCATGGTAGGAGTC | 79 | PGK1 | phosphoglycerate kinase 1 |
| TTCAACGCTTTCGGAATGAGTTTGA | 80 | PHYH | phytanoyl-CoA 2-hydroxylase |
| GAGGAAGAAGACCATCTGTGGCACC | 81 | PLK3 | polo like kinase 3 |
| TTCCTCATCACCCAGATCCTGGTGG | 82 | PRKD2 | protein kinase D2 |
| GGCGGCCGCGGGATCTCCCAGCCAT | 83 | PROSC | proline synthetase cotranscribed homolog |
| | | | (bacterial) |
| GAATCAGCCGCCAGATCCCACCCTT | 84 | RAB31 | RAB31, member RAS oncogene family |
| GAGATCGATGAGCATGCCCCCGGAG | 85 | RAB40B | RAB40B, member RAS oncogene family |
| GCACTCCCAAGATTGATAATTAGGA | 86 | RBPJ | recombination signal binding protein for |
| | | | immunoglobulin kappa J region |
| GGCTGCTTCTCATAGGGCACTTGAT | 87 | REXO2 | RNA exonuclease 2 |
| AGCCGCTTCTGGAAGCATGAGTGGG | 88 | RNASET2 | ribonuclease T2 |
| ATACCTCAGAGCTGGCGGCTGTACC | 89 | RNF213 | ring finger protein 213 |
| GCCCTGAATGATTTTTGCTGCAATT | 90 | RPS16P5 | ribosomal protein S16 pseudogene 5 |
| GAGCATAGTCTAGGGGTGGGGTATC | 91 | SAMHD1 | SAM and HD domain containing |
| | | | deoxynucleoside triphosphate |
| | | | triphosphohydrolase 1 |
| GCTCGGGCTACTTCGAGCAGGAGTC | 92 | SDC3 | syndecan 3 |
| TCTTCTTTAAAGGCAAATGGGAGAG | 93 | SERPINA1 | serpin family A member 1 |
| AGCCTGAAAGTGAAAGCAAAATCTA | 94 | SIRPB2 | signal regulatory protein beta 2 |
| GGAACAACAGGCCTTATTCCACTGC | 95 | SOD2 | superoxide dismutase 2, mitochondrial |
| ATGAAGAAAGCCCAGAGGCAGAGCT | 96 | SP100 | SP100 nuclear antigen |
| TATTCACCGAAGAGGAAAACCCAAA | 97 | SP110 | SP110 nuclear body protein |
| AGGACAAGGAGATGGGAATGGAAAT | 98 | SPATA13 | spermatogenesis associated 13 |
| AACCCTGGGAGAAAGTCAGCGGACA | 99 | SRGAP2 | SLIT-ROBO Rho GTPase activating |
| | | | protein 2 |
| CTCAGCTCCTGCAGCACCCATTTGT | 100 | STK4 | serine/threonine kinase 4 |
| TATAAAAGACCTATCCCTCTTTGTT | 101 | THEM4 | thioesterase superfamily member 4 |
| TCTCATTGCAGGAAAGGCCGAGGGG | 102 | TIMP2 | TIMP metallopeptidase inhibitor 2 |
| AACGTTTGGGTTTTCCTAGCTACAT | 103 | TMEM14C | transmembrane protein 14C |
| CTCCTGTAGTAACTGTAAGAAAAGC | 104 | TNFRSF1A | TNF receptor superfamily member lA |
| TTTCCCATGGATTCCTTGGCAGCAG | 105 | TRAK1 | trafficking kinesin protein 1 |
| CAGGTCCCACTCAGGAATCCTCAAT | 106 | TRANK1 | tetmtricopeptide repeat and ankyrin repeat |
| | | | containing 1 |
| GAGGTGGTCCAAGCCCAGGAAGCGC | 107 | TSHZ1 | teashirt zinc finger homeobox 1 |
| ACTGTGGCTGAGCATCTCTGGAAGT | 108 | TUBGCP4 | tubulin gamma complex associated |
| | | | protein 4 |
| CGGCCTCCAAGCGGCCGTGGTGTGT | 109 | UPP1 | uridine phosphorylase 1 |
| ACGCGGGGCGCAGTGGTATCTTATT | 110 | USP4 | ubiquitin specific peptidase 4 |
| CCTACCTCCCAGCCTAATTGACCGG | 111 | ZDHHC18 | zinc finger DHHC-type containing 18 |
| GTTACAAGTGTGAGGACTGCGGGAA | 112 | ZYX | zyxin |

### Gene Sequences

Gene Sequences

SEQ ID NO: 1
ctcggtgcgg ttccgccggg cgcgaggagc cgccgagacc tccgcctgcg aacaaagagg
aggccgtgcg gggcgcggcg cccgcggagc atggcggacc gcagcctgga gggcatggcg
ctgcccctgg aggtgcgggc gcgcctggcc gagctggagc tggagctgtc ggaaggtgac
atcacacaaa aaggatatga aaagaagagg tcaaagttaa ttggagccta ccttccgcag
cctccgaggg tggaccaagc tttgccgcaa gaacgccggg ctcctgtcac tccttcctcc
gcctctcgct accaccgccg acggtcttca gggtcacgag atgagcgcta tcggtcagac
gtccacacgg aagctgtcca ggcggctctg gccaaacaca aagagcggaa gatggcagtg
cctatgcctt ccaaacgcag gtccctggtc gtgcagacct cgatggacgc ctacacccct
ccagatacct cttctggctc agaagatgaa ggctcagtgc agggggactc ccagggcacc
cccacctcca gccagggcag catcaatatg gagcactgga tcagccaggc catccacggc
tccaccacgt ccaccacctc ctcgtcctct acgcagagcg ggggcagcgg ggctgcccac
aggctggcgg acgtcatggc tcagacccac atagaaaatc attctgcacc tcctgacgta
accacgtaca cctcagagca ctcgatacag gtggagagac cgcagggttc cacggggtcc
cggacagcgc ccaagtacgg caacgccgag ctcatggaga ccggggatgg agtaccagta
agtagccggg tgtcagcaaa aatccagcag cttgtcaata ccctcaaacg accgaaacga
ccacctttac gagaattctt tgtcgatgac tttgaagaat tattagaagt tcaacaaccg
gatccgaacc aaccaaagcc ggagggggcc cagatgctgg ccatgcgcgg agagcagctg
ggcgtggtca cgaactggcc gccgtcgctg gaggccgcac tgcagaggtg gggcaccatc
tcgcccaagg cgccctgcct gaccaccatg gacaccaacg ggaagcccct ctacatcctc
acttacggca agctgtggac aagaagtatg aaggtcgctt acagcattct acacaaatta
ggcacaaagc aggaacccat ggtccggcct ggagataggg tggcactggt gttccccaac
aatgatccgg ctgccttcat ggcggctttc tacggctgcc tgctggccga ggtggtcccc
gtgcccatcg aggtgccgct caccaggaag gacgcaggga gccagcagat aggtttcttg
cttggaagct gtggagttac tgtagccttg actagtgacg cctgccataa aggacttcca
aaaagcccaa cgggagagat cccacagttt aaaggttggc caaagctgct gtggtttgtc
acagagtcta aacatctctc caaaccgccc cgagactggt tcccacacat taaagatgcc
aataacgaca ctgcgtatat tgagtacaag acgtgtaagg atggcagtgt gctgggtgtg
acggtgacga ggactgcgct gctgacacac tgccaggccc tgacgcaggc gtgtggctac
acggaagctg aaaccattgt gaatgtgctg gatttcaaga aggacgtcgg gctctggcat
ggcatcctga caagcgtcat gaacatgatg catgtgatca gcatcccgta ctcgctgatg
aaggtgaacc ctctctcctg gatccagaag gtctgccagt acaaagcaaa agtggcgtgt
gtgaaatcga gggatatgca ttgggcatta gtagcacaca gagatcagag agacatcaac
ctctcctctc tgcgaatgct gatagtggcg gacggcgcga acccctggtc tatttcttct
tgcgatgcat ttctcaatgt cttccaaagt aaaggccttc gacaggaggt catctgtcct
tgtgccagct cgccagaggc cctcactgtg gccatccgga ggcccacgga tgacagtaac
cagcccccgg gccggggtgt cctctccatg catggactga cctatggggt cattcgtgtg
gactcggaag agaagctgtc cgtgctcacc gtgcaggatg tcggcctcgt gatgcctgga
gccatcatgt gttcagtgaa gccagacggg gttcctcagc tgtgcagaac ggatgagatc
ggggagctgt gtgtgtgtgc agttgcgacg ggcacgtcct actatggcct ctctggcatg
accaagaaca cctttgaggt gtttcccatg acaagctccg gggctccgat cagtgaatac
ccattcataa ggacaggctt gctggggttc gtgggtcccg gaggcctcgt cttcgtggtg
ggcaagatgg atggcctcat ggtggtcagc gggcgcaggc acaacgccga cgacatcgtg
gccactgcgc tggccgtaga acccatgaag tttgtctacc ggggaaggat agccgtgttc
tcggtgaccg tgctgcacga cgagaggatc gtgatcgtgg ctgagcagag gcctgactcc
acggaagagg acagtttcca gtggatgagc cgtgtgctgc aggcgattga cagtatacat
caagttggag tttattgcct ggccttggtg ccagcaaaca ccctccccaa aaccccgctt
ggtgggatcc atttatcaga aacaaaacag ctttttctgg agggctctct gcacccctgc
aatgtcctaa tgtgccccca cacctgcgtc acaaacttgc ctaagcctcg acagaagcag
ccagaaatcg gccctgcctc tgtgatggtg gggaacctgg tctctgggaa gagaatcgcc
caggccagtg gcagagacct gggtcagatc gaagataacg accaggcacg caagttcctg
ttcctctcag aggtcttgca gtggagagca cagaccaccc cggaccacat cctctacacg
ctgctcaact gtcggggtgc gatagcgaac tcgctgacct gcgtgcagct gcacaagaga
gctgagaaga tcgccgtgat gctgatggag aggggccacc ttcaggacgg cgaccacgtg
gccttggtct accccccagg aatagacctg atagcagcgt tttatggttg cctgtacgca
ggctgtgtgc caataaccgt ccgtcccccg cacccacaga acatcgcgac gacgttgcct
accgtcaaga tgattgtgga ggtgagtcgc tctgcctgtc tgatgacgac acagctgatc
tgtaagttgc tgcggtccag ggaggcggcg gcggctgtgg acgtcaggac gtggcccctc
atcctggaca cagatgattt gccaaagaag cggcctgccc agatctgcaa accttgcaac
ccagacactc ttgcatatct cgacttcagc gtgtccacaa ctgggatgct agctggcgta
aagatgtctc acgcagccac cagtgccttc tgccgttcca ttaagctgca gtgtgaactt
tacccctcta gagaagtggc catctgcctg gacccttact gtggactggg atttgtcctc
tggtgcctct gcagtgtgta ttctgggcac cagtccatcc tgatcccgcc ctctgagctg
gaaaccaacc ccgccttgtg gcttcttgcc gtgagtcagt acaaagtccg agacacgttt
tgctcctact ccgtgatgga gctgtgcacc aaggggctgg gctcgcaaac agagtccctc
aaggcgcgag ggctggactt gtcccgagtg aggacctgcg tggttgtggc ggaagagagg
cctcggatcg cactcacaca gtcgttctca aagctgttta aggacctggg ccttcacccg
cgggccgtca gcacctcgtt cggttgcagg gtgaacctgg cgatttgctt gcagggaacc
tcaggacctg acccaaccac tgtctacgtg gacatgagag ccctgagaca cgacagagtc
cgcttagtgg aaagaggatc ccctcatagt ctgcccctga tggaatcggg aaagatactt
ccaggggttc ggattataat tgccaaccca gaaacaaaag gaccgctggg ggactcacac
cttggagaga tttgggttca cagtgcccac aatgccagcg gttatttcac tatttacgga
gacgaatccc tccagtcaga tcacttcaac tcaagactaa gttttggaga cacccagacc
atctgggcac gcacaggcta cttggggttc ctgcggagaa ctgagctcac agatgcaaat
ggagagcgcc atgatgccct ctacgtggta ggggcactgg acgaagccat ggagctgcgg
ggcatgcggt accacccaat cgacattgag acctcggtca tcagagccca taaaagcgtt
acggaatgtg ctgtgtttac ctggacaaat ttgttggtgg ttgtggttga gctggatggg
tcggaacaag aagccttgga cctggttccc ttggtgacca acgtggtcct ggaggagcac
tacctgatcg tcggagtggt ggtcgtggtg gacatcggcg tcatccccat caactcccgt
ggggagaagc agcgcatgca cctgcgagac gggtttttgg cagaccagct agaccccatc
tatgtggcct acaacatgta gtctcgtctc ttggcttcca tggacttttc tagagatgta
gacattgttc tccgtgtcca ctgaagcgtg cagacacagg gcaacactca ccagaataca
gccatttgtg gtgagagtgg aggaggaaga ggaggaggaa gaggacttct cacagcagcc
acgattggca tgggggtgaa atgtgaattt accactgaat ttcgctcaga aggactttgg
attactgcct tcagtttgtt ggaaaagccc atttcaaaac tttcttttct tttctttctt
ttttaattat tggataataa gtgctttctt cgtaaatgtg gtattttgtt aagccgaaat
agcaattaaa aaaatatcct gccctccaga tgggttcttt taaacaattt atgtagtgtg
acaaagaatt gttttctctg ttttaatgtg tcatgaaatc ttaatgacat ggatctgtta
ctaatttaag ccattgctag atctcatcct tttaggaaag tttgaggtac gagaaaacct
tccaaatagc accttccaat tagataatag cagctttctt tgtcagaaat gtgctgaaga
aacaaaggct ggtatacggc cttcgaagtt agtatagaat gagaagaaat tataaataag
gtgtatttcg gcaattatct tgcaaatatc tttgtactaa actaaaaaga taaaataagt
taacttcctc aatatgtaat tatgtacaaa acgtttaatt tattttgatc tctttagaac
tataaaagag aaaaacattc aagaatatta aagtcttgta atgtttgcta atataaaaaa
gtgttgtatt atcttgcgtg gatagtatca caacaaatat atatatatga aatataaatt
cactaatgaa caaaggagat tttaaagttt aagatgcaga acttgtcact tgcatggtgt
gccccccgta ctcacataca ctctgctgtt gccagcagtc gcagaccgca ggagccctgt
ctaaaagttt cttctagaac cagagaccag caagtgaaat tattgccatc tcaaggatgg
caaaagaatt caaagctcaa tgtgcactat ttttttcttt gctgtgggac aacagtgaat
gtgtttatgc cagcgtgtgc tgatgatact gaggggcttt aggttggcaa atagcactgt
tttcttagct gcaagaattc attgcacaat gtttttcatc atttttgtta atgtcatctt
tttttggtcc ttgctacgaa aaggaatgcg attctgtggt cattcgcact gggttgcatt
gattccccct ctgatggcca atgtggagtg gacaaagtgt ccggaactca catcggtgat
cgtcccctcg tcttaagacc cagcccgctc tgtgtgagcc tctggggctc cctcgctcag
tgagcacagt tccccggggg ttcatgccag agctccggct gaagcaagaa gtcctccagc
tgcgtcgttt gccgcctgtg gacgagtgcg ccccagtttc tgccctggca gctcctggcc
acaccttctc agagctcacc tgtgcacttc taaattgaat tggcccacgg tgtccaacca
agaaggagca tctgcactcc gagaaagatg tgttctgtaa ctgccccagt gtgaccccgc
agtggctctc ggtgctagat atgcatgact aagattgatg ctgggcaaaa tgtagatgat
ctttcattat gttgtgggca gcgtctttct ctgcctttgc tatatgcagt cagcagtaag
ccttttgcta aaagagtttt gtttgacttc tgagatccaa ggctgattgt tgttaaaaaa
aaaaaaaaaa aaaaagtggc acatttaaaa aaatgtgtct gcatatgtgg tgcatccttc
catctccaca aaccatttga ttcttgaaat attgtttgac ctcattgctg tgtgtgaata
tttctccaca tgcttcagat gcacattcct agtctctgct tcctaagggg ggaaccacca
cacattgggg ggaaaaaaga cattttccta cacccaccca ccttgttgaa agggaggtag
gtttggggct tcaggccagg cactgactat gaaacattag ctgcagtgtg caggacagct
ttgaggtcca gctgaagtca ggaagcaaaa caaatgtaga tgtcacttca aacataattt
caactgtcac cagatcaact ctacattcaa ggagtgtgga cgctgcagtg cagttgtgag
ggcagttagc agccgcctct tctgcatcct gtcaactctg attagttaga gtttaggctc
aaaagagttg gtggactgag attgaaattt ggttgtgcaa gagaaaggaa aggagacact
tagtaccacc agtttcagca ataaagaagg gtcattctgt attcaaaatt gtactgtaga
taaatcattc atgagattgt aaaaaatgtt tgtcttgtga ccttgtgctt ttgaagtcag
acaaaaccgt gtaatcaact tgcacaaaaa gagggtacac agtgaacata taaacacaga
cctaatcaaa caggagcaga ttcctcatgg tgcttgttta ttatatatat ttaatcctgc
ttgacacttt acccaaggga gatggtccct tttatcagtt gaatgttagc agcgttattt
cagagtgtgg tgactggtta gagaaactca tgtactcaac cagccacagt ttcaaacaaa
atttttatgt gcaaaggaca gcaaccttct tgtatgttaa accaccagta cgctttgtac
atctgtgata acgcctgttt tatattcaaa tgaacaaata aaagctttta tttttgttgc
tctgaaaata gcagtttctt aattggtccc ctggaaagat gtctgggaca gctttaatcc
cgggaaggaa gtgactccta cagggaaatg tatctgactc tgtttacata atttgttgca
ttacttagta cagataatca tactttgaaa aatgtttaaa ttttgatgtg ggcatttatt
gctaaaaata attcctatgg caacaaatgt tttgtgaaat gtttttttta attcttttaa
atatatctaa atatatttgt tcaca
SEQ ID NO: 2
MADRSLEGMALPLEVRARLAELELELSEGDITQKGYEKKRSKLI
GAYLPQPPRVDQALPQERRAPVTPSSASRYHRRRSSGSRDERYRSDVHTEAVQAALAK
HKERKMAVPMPSKRRSLVVQTSMDAYTPPDTSSGSEDEGSVQGDSQGTPTSSQGSINM
EHWISQATHGSTTSTTSSSSTQSGGSGAAHRLADVMAQTHIENHSAPPDVTTYTSEHS
IQVERPQGSTGSRTAPKYGNAELMETGDGVPVSSRVSAKIQQLVNTLKRPKRPPLREF
FVDDFEELLEVQQPDPNQPKPEGAQMLAMRGEQLGVVTNWPPSLEAALQRWGTISPKA
PCLTTMDTNGKPLYILTYGKLWTRSMKVAYSILHKLGTKQEPMVRPGDRVALVFPNND
PAAFMAAFYGCLLAEVVPVPIEVPLTRKDAGSQQIGFLLGSCGVTVALTSDACHKGLP
KSPTGEIPQFKGWPKLLWFVTESKHLSKPPRDWFPHIKDANNDTAYIEYKTCKDGSVL
GVTVTRTALLTHCQALTQACGYTEAETIVNVLDFKKDVGLWHGILTSVMNMMHVISIP
YSLMKVNPLSWIQKVCQYKAKVACVKSRDMHWALVAHRDQRDINLSSLRMLIVADGAN
PWSISSCDAFLNVFQSKGLRQEVICPCASSPEALTVAIRRPTDDSNQPPGRGVLSMHG
LTYGVIRVDSEEKLSVLTVQDVGLVMPGAIMCSVKPDGVPQLCRTDEIGELCVCAVAT
GTSYYGLSGMTKNTFEVFPMTSSGAPISEYPFIRTGLLGFVGPGGLVFVVGKMDGLMV
VSGRRHNADDIVATALAVEPMKFVYRGRIAVFSVTVLHDERIVIVAEQRPDSTEEDSF
QWMSRVLQAIDSIHQVGVYCLALVPANTLPKTPLGGIHLSETKQLFLEGSLHPCNVLM
CPHTCVTNLPKPRQKQPEIGPASVMVGNLVSGKRIAQASGRDLGQIEDNDQARKFLFL
SEVLQWRAQTTPDHILYILLNCRGAIANSLICVQLHKRAEKIAVMLMERGHLQDGDHV
ALVYPPGIDLIAAFYGCLYAGCVPITVRPPHPQNIATTLPTVKMIVEVSRSACLMTTQ
LICKLLRSREAAAAVDVRTWPLILDTDDLPKKRPAQICKPCNPDTLAYLDFSVSTTGM
LAGVKMSHAATSAFCRSIKLQCELYPSREVAICLDPYCGLGFVLWCLCSVYSGHQSIL
IPPSELETNPALWLLAVSQYKVRDTFCSYSVMELCTKGLGSQTESLKARGLDLSRVRT
CVVVAEERPRIALTQSFSKLFKDLGLHPRAVSTSFGCRVNLAICLQGTSGPDPTTVYV
DMRALRHDRVRLVERGSPHSLPLMESGKILPGVRIIIANPETKGPLGDSHLGEIWVHS
AHNASGYFTIYGDESLQSDHFNSRLSFGDTQTIWARTGYLGFLRRTELTDANGERHDA
LYVVGALDEAMELRGMRYHPIDIETSVIRAHKSVTECAVFTWTNLLVVVVELDGSEQE
ALDLVPLVTNVVLEEHYLIVGVVVVVDIGVIPINSRGEKQRMHLRDGFLADQLDPIYV
AYNM
SEQ ID NO: 3
agtcctgacc gcacgggggc cgcggccacg gggcgcaggg gccatggtgc gcggcaggat
ctcccggctc tcggtccggg acgtgcgctt ccccacgtcg cttgggggcc acggcgcgga
cgccatgcac acggaccctg actactcggc tgcctatgtc gtcatagaaa ctgatgcaga
agatggaatc aaggggtgtg gaattacctt cactctggga aaaggcactg aagttgttgt
ctgtgctgtg aatgccctcg cccaccatgt gctcaacaag gacctcaagg acattgttgg
tgacttcaga ggcttctata ggcagctcac aagtgatggg cagctcagat ggattggtcc
agaaaagggc gtggtgcacc tggcgacagc ggccgtccta aacgcggtgt gggacttgtg
ggccaagcag gagggaaagc ctgtctggaa gttacttgtg gacatggatc ccaggatgct
ggtatcctgc atagatttca ggtacatcac tgatgtcctg actgaggagg atgccctaga
aatactgcag aaaggtcaaa ttggtaaaaa agaaagagag aagcaaatgc tggcacaagg
ataccctgct tacacgacat cgtgcgcctg gctggggtac tcagatgaca cgttgaagca
gctctgtgcc caggcgctga aggatggctg gaccaggttt aaagtaaagg tgggtgctga
tctccaggat gacatgcgaa gatgccaaat catccgagac atgattggac cggaaaagac
tttgatgatg gatgccaacc agcgctggga tgtgcctgag gcggtggagt ggatgtccaa
gctggccaag ttcaagccat tgtggattga ggagccaacc tcccctgatg acattctggg
gcacgccacc atttccaagg cactggtccc attaggaatt ggcattgcca caggagaaca
gtgccacaat agagtgatat ttaagcaact cctacaggcg aaggccctgc agttcctcca
gattgacagt tgcagactgg gcagtgtcaa tgagaacctc tcagtattgc tgatggccaa
aaagtttgaa attcctgttt gcccccatgc tggtggagtt ggcctctgtg aactggtgca
gcacctgatt atatttgact acatatcagt ttctgcaagc cttgaaaata gggtgtgtga
gtatgttgac cacctgcatg agcatttcaa gtatcccgtg atgatccagc gggcttccta
catgcctccc aaggatcccg gctactcaac agaaatgaag gaggaatctg taaagaaaca
ccagtatcca gatggtgaag tttggaagaa actccttcct gctcaagaaa attaagtgct
cagccccaac aacttttttc tttctgaagt gaaagggctt aaaatttctt ggaaatagtt
ttacaaaaat ggatttaaaa aatcctaccg atcaagatga gttcagctag aagtcatacc
accctcagga atcagctaag taattattac ttgattcttt tagcaaatca atgcacgtta
tcctacttaa tccttaaata agtttagatt taactaaccc aaagtccagg aggatgttct
tacaaaaata gctatatcaa gggctggcac ctagacatta aactgtaatt tgaaaataag
caacatgttg cataacttgt tggaataatt ccttgttctg tttaacactt gtcataaatt
agcagaataa aaatagtcgt gcaacaccgg gggtatctgg tatgcaacga agggaaaaat
atttcactga ttaaccccga agtggttttg catcttttcc ttgcttaatc taagcatatt
attagagaag tcacaccatg ctgaagctaa tgagggcaaa atggtagtcc atagattatt
ttaaaataac cctttaaggt tataaaagtt taaaaaaaaa aaaaaaaaac tctatcctaa
atggtcatta tattttgagg ataagatgca gttaaaatga gaaaaatagg gcaaaatata
ttcactatta tttctaaaat atactctttt aagtagcatc caaaccagaa tacagcacat
gtttacttaa ggagagttct ttaatctatt ttaggaagga actgagcaga taagtggcag
tacagaatga acaaagcgtg gacgaatgca gaacacttct ttattatagc aacatataaa
acaactataa ctttaaagtt cataaccaca ctctacatca tgatcgatgg tgttactcag
ctccctcaga tttgagggaa tagcttgtga aattcttaaa atattctaaa aatattccaa
aaatagcttg tgaaattcac caaccttctt tataagtacg tgggattgaa atgcacatac
atgtttttgc taagagcaca tacatttcat tctcctcact ttgttcataa cctcagcatt
gtcagatacc ctcagtgagt taactcaaag ccttttatta tggaaagaac tggcacagtt
acatttgcca gtggcaacat ccttaaaaat taataactga taggtcacgg acagattttt
gacctagttc ctttttcttt tagagcaaaa agaactttta cctcggcatc cagcccaacc
cctaaagact gacaatatcc ttcgagctcc tttgaaagca ccctaaacag ccatttccat
tttaatagtt ggatgcggat tgtacccttc aatctgaaag tcttcagctt tgaagtcatc
aattttctca acttttcgaa gaatcctgag ctttgggaaa ggtctgggtt ctcgctgaag
ctaaaaacaa aataaggcca ttattttgcc ataattgtac gacctgttgt aattgctcct
catgtccgtg aaacaagtac acaggatgtg atcaacaaag ttctatttta caggagtatg
atcctgtcga taccttgccg taggttatgt aacatgattg gagcgcaacc agctgttctc
ttgcacagat cgagagtgag gggtattttg tgacattaca cagcatcagg agcctggtgc
ctcatcaggt gtaagttctt ataaccactc ttggcaaatt tattaaagac aggaacacag
tcaatctgta actcatagta gctctacgtt tacttgaatt ccacaatccc taacccatct
gtccctggca gaaagaagga aagatgacat gcatggacag tgaacagaaa gggatgaaag
ccaggattcc tgggatgaac agacagtggc aattaggatg tgaagacagg tcacaaccta
ttactatgtc taaaaacgac cagagcagag agccagagag aataagcctg aagtcacctc
cactcaaaag cagccaaact ccctcaaagg agtaactttt aaaacctgga tctaacctgg
aaggggctaa aaagtgtctg gttctgagtt tttttcctta aggctcatga agcagatgaa
cttacatttt tattgccatt tcatatcaat tgttggctgc tataacttag ggatttcaac
agacttttga agtttggacc taaatattgt acttaatgta aaattaacaa aaaatattta
tggccagggt ggtggcttat gcctgtaatt ccagaatttt cggaggctga ggcaggtgga
tcacttgaag tcaggagttt gagactagcc tggccaacat gatgaaaccc catctctact
aataatacaa aaattagctg ggtgtggtgg catgtgcctg taatcccagc tacctgggag
gctgaggcag aagaattgct tgaacccggg aggtggaggt tgcagtgagc tgagatcgca
ccacggcaca ctccagcctg gccgacagag aaagactcca tctcaaaaaa aaaagaaaag
gaaaaacatt tgcacttcaa ttctccttca agttaaaatg agttaaaatg cccccttttg
gacaatcccc tggcttgaat gtggctcttc cctctctggt actggtgctt agtacctcac
agcacctgac atgttaagtg cccatggttg ctgaggcaga tgcctgcctt gtcctgccca
cctgcccacc acttctccct aaactgaagc cccacatttg gagcagtcat ctttatcttg
gacacagcat tgagcagatg cctgttccac agtcaacctt ttatcaagag aaggtaccaa
acccaaaagt ataacatcta attcttacct gaattttcag tggctcgatg tgattcaggt
aaatatgtgc atctcccaaa gtgtgtataa agtcacctgg ctataaaccc gggggagaaa
gcagaacagt atgttagttt caattcttta aaacatcatt taaaaacatt agaatatgca
gacaccgcaa ggcttttttt aaaaaaataa tttagtgtag cttttccatt tttttgtagc
aacagcatct tgttatgttg cccaggctgg tattgaactc cagacctcaa gcaattgctc
ctgtctcagt ctcccaaagt gctgggatta caggcatgag ccaccatacc caacctcagc
atagcttttg agaaaatcca tagaagctgt atcacaaaca acctgtatag atctgttagt
gcgtatacca cagggccaga aaaccttcca gaagaggaag gtttcaaagt aaaagctggt
tcatttctta cttacacata tcaaatttaa aagctaatca gagactaaac tctgcaattt
gttttcccat attaaagaac tgaagagctc agtgtggtag gctggcaagt cacccttccc
gagacagccc accttcaggc ccgtgatgtg cgcaatcatg tacgtgagca gggcgtagct
ggcgatgttg aaaggcacac cgaggcccat gtctcccgat ctctggtaca gctggcagga
cagctcactg ttcaccacat agaactggca gagggcatgg catggaggca gcgccatcag
aggaagatct gaggaaccag cagaggaaga taaggaggga tggtggtttg aaagaccaca
gctaaaggca aagtaaaaca ggagagaaac agaagccaac tcatatggtg gagaccagga
gagagagcca ctgggctgca gtgatgtcca taacagcctc tgcagcgatg gcacggagct
gagggagact atccatcggt gcaaggtttc tgcaggtgtc catttacggc tgaagcaatg
ctcttccatc agagctgaag ggatctgggc tacctcgtgg caccagatta caaatacagc
aggaataatt ctgtttgcca caggaaactg gtgcttctgg tacaccctcc tatattaaaa
gtctctatta catggccagg ca
SEQ ID NO: 4
MVRGRISRLSVRDVRFPTSLGGHGADAMHTDPDYSAAYVVIETD
AEDGIKGCGITFTLGKGTEVVVCAVNALAHHVLNKDLKDIVGDFRGFYRQLTSDGQLR
WIGPEKGVVHLATAAVLNAVWDLWAKQEGKPVWKLLVDMDPRMLVSCIDFRYITDVLT
EEDALEILQKGQIGKKEREKQMLAQGYPAYTTSCAWLGYSDDTLKQLCAQALKDGWTR
FKVKVGADLQDDMRRCQIIRDMIGPEKTLMMDANQRWDVPEAVEWMSKLAKFKPLWIE
EPTSPDDILGHATISKALVPLGIGIATGEQCHNRVIFKQLLQAKALQFLQIDSCRLGS
VNENLSVLLMAKKFEIPVCPHAGGVGLCELVQHLIIFDYISVSASLENRVCEYVDHLH
EHFKYPVMIQRASYMPPKDPGYSTEMKEESVKKHQYPDGEVWKKLLPAQEN
SEQ ID NO: 5
ggtacgcgga caagatggcg gcggcagcag tcgacagcgc gatggaggtg gtgccggcgc
tggcggagga ggccgcgccg gaggtagcgg gcctcagctg cctcgtcaac ctgccgggtg
aggtgctgga gtacatcctg tgctgcggct cgctgacggc cgccgacatc ggccgtgtct
ccagcacctg ccggcggctg cgcgagctgt gccagagcag cgggaaggtg tggaaggagc
agttccgggt gaggtggcct tcccttatga aacactacag ccccaccgac tacgtcaatt
ggttggaaga gtataaagtt cggcaaaaag ctgggttaga agcgcggaag attgtagcct
cgttctcaaa gaggttcttt tcagagcacg ttccttgtaa tggcttcagt gacattgaga
accttgaagg accagagatt ttttttgagg atgaactggt gtgtatccta aatatggaag
gaagaaaagc tttgacctgg aaatactacg caaaaaaaat tctttactac ctgcggcaac
agaagatctt aaataatctt aaggcctttc ttcagcagcc agatgactat gagtcgtatc
ttgaaggtgc tgtatatatt gaccagtact gcaatcctct ctccgacatc agcctcaaag
acatccaggc ccaaattgac agcatcgtgg agcttgtttg caaaaccctt cggggcataa
acagtcgcca ccccagcttg gccttcaagg caggtgaatc atccatgata atggaaatag
aactccagag ccaggtgctg gatgccatga actatgtcct ttacgaccaa ctgaagttca
aggggaatcg aatggattac tataatgccc tcaacttata tatgcatcag gttttgattc
gcagaacagg aatcccaatc agcatgtctc tgctctattt gacaattgct cggcagttgg
gagtcccact ggagcctgtc aacttcccaa gtcacttctt attaaggtgg tgccaaggcg
cagaaggggc gaccctggac atctttgact acatctacat agatgctttt gggaaaggca
agcagctgac agtgaaagaa tgcgagtact tgatcggcca gcacgtgact gcagcactgt
atggggtggt caatgtcaag aaggtgttac agagaatggt gggaaacctg ttaagcctgg
ggaagcggga aggcatcgac cagtcatacc agctcctgag agactcgctg gatctctatc
tggcaatgta cccggaccag gtgcagcttc tcctcctcca agccaggctt tacttccacc
tgggaatctg gccagagaag tctttctgtc ttgttttgaa ggtgcttgac atcctccagc
acatccaaac cctagacccg gggcagcacg gggcggtggg ctacctggtg cagcacactc
tagagcacat tgagcgcaaa aaggaggagg tgggcgtaga ggtgaagctg cgctccgatg
agaagcacag agatgtctgc tactccatcg ggctcattat gaagcataag aggtatggct
ataactgtgt gatctacggc tgggacccca cctgcatgat gggacacgag tggatccgga
acatgaacgt ccacagcctg ccgcacggcc accaccagcc tttctataac gtgctggtgg
aggacggctc ctgtcgatac gcagcccaag aaaacttgga atataacgtg gagcctcaag
aaatctcaca ccctgacgtg ggacgctatt tctcagagtt tactggcact cactacatcc
caaacgcaga gctggagatc cggtatccag aagatctgga gtttgtctat gaaacggtgc
agaatattta cagtgcaaag aaagagaaca tagatgagta aagtctagag aggacattgc
acctttgctg ctgctgctat cttccaagag aacgggactc cggaagaaga cgtctccacg
gagccctcgg gacctgctgc accaggaaag ccactccacc agtagtgctg gttgcctcct
actaagttta aataccgtgt gctcttcccc agctgcaaag acaatgttgc tctccgccta
cactagtgaa ttaatctgaa aggcactgtg tcagtggcat ggcttgtatg cttgtcctgt
ggtgacagtt tgtgacattc tgtcttcatg aggtctcaca gtcgacgctc ctgtaatcat
tctttgtatt cactccattc ccctgtctgt ctgcatttgt ctcagaacat ttccttggct
ggacagatgg ggttatgcat ttgcaataat ttccttctga tttctctgtg gaacgtgttc
ggtcccgagt gaggactgtg tgtcttttta ccctgaagtt agttgcatat tcagaggtaa
agttgtgtgc tatcttggca gcatcttaga gatggagaca ttaacaagct aatggtaatt
agaatcattt gaatttattt ttttctaata tgtgaaacac agatttcaag tgttttatct
ttttttttta aatttaaatg ggaatataac acagttttcc cttccatatt cctctcttga
gtttatgcac atctctataa atcattagtt ttctatttta ttacataaaa ttcttttaga
aaatgcaaat agtgaacttt gtgaatggat ttttccatac tcatctacaa ttcctccatt
ttaaatgact acttttattt tttaatttaa aaaatctact tcagtatcat gagtaggtct
tacatcagtg atgggttctt tttgtagtga gacatacaaa tctgatgtta atgtttgctc
ttagaagtca tactccatgg tcttcaaaga ccaaaaaatg aggttttgct tttgtaatca
ggaaaaaaaa aaattaatga accttaaaaa aaaaaaaaaa ggttttgaag ggaaaaaaag
tggtttcaca cctcttgtta ttccttagag tcacttcaag gcctgtttga atgtggcagg
ttagaaagag agagaatgtc tttcatttga agagtgttgg acttgtgtga aaggagatgt
gcgtgttgga atctgctttt ccaagccgcc agggtcctga cggcagcagg acgaagcctg
ttgtggcgtc ttctgggaaa gcctgaccgt gtgttcggac ggcactggct cctttccgaa
gttctcagta actgagccca gagtaactgc acgcctttgt gcagctctgg agctccacca
actctcggcc tgccagttct caagcgagct aatcttgtca ttaatcgata gaagctaact
tccgaagtta ggacctagtt actttgctct caacatttaa aataatgcag ttgctctagt
gaatggggcg ttaggggcct gtctctgcac ctgtctgtcc atctgcatgc agtattctca
cccatgttga atgcctgctg cttgtttacc ctttggaaac cctggggtga ccaaggtttg
gaaagccacc tgagaccact tcatagcaag ggaaggcttt aagcagttac tagaaagaga
tggggatttg gcccctggct cctccagcct gaatgagcta tttaatccac tgtccatgtt
cctcatcagt caaatccaaa gtcaaaggat ttgaacctgc atctggaaac gtaaccactc
acagcacctg gcccgccaag gttgggagga ttgtacacta ctttcattta aaggggaaag
tttgataata cggaattaat taatatgaat gagatgcatt aataagaacc tgagcatgct
gagagttgca attgttggtt ttctggtttg attgatttcc ttttttctta gacacatcaa
agtcaagaaa gatggtttta cctttactga cccagctgta catatgtatc tagactgttt
ttaaatgtct ttcttcatga atgcttcatg gggctccagg aagcctgtat cacctgtgta
agttggtatt tgggcacttt atatttttct aaaaacgtgt tttggatcct gtactctaat
aaatcataag tttcttttta aaaattttcc aaaacttttc tccattttaa aaagccctgt
tataaacgtt gaactttcac aatgttaaaa tgttaaatat ttggatatag caacttcttt
tctcttcaaa tgaatgccaa gatttttttg tacaatgatt aataaatgga acttatccag
agaaaccacg caaatggcct gcccaatttc gtttgaggac agaaagccca gccatgactt
gagtagaatg tctctcacct ctcttcggat ctaaatatga aaagtatgtt ctgctgaatt
tttctgagca ttggtgagcg gacagcctac ctgtaaacca tgacctcctt gccaaacgtt
aattttatca gctcactagt aacctttgag aattatctgg ttgtcatgca aagattgcac
tttctgaatt atgttaaaac acatgttgta aaatgagaac tgctcatgct ttgaaagaaa
accaggttct tcgtgcgttc tgttgccgtt gatttgaatg gctgtgctgt atacgatgtg
tccagaatgt cttcagagca ctgtttccgt gtgatgttac tacctactat gtgggaggaa
aaaaggttat aggttaaaca aagtcaattg actctatggt ggtgtttctc aatacctgtg
acgcacagta ctgtgcgtcg tgactttcta agagaagtgt gcagcgggtg tgtcatcttg
atatatgaaa ccctggaatt tccctcccct acacgcacgc accgtccccg ggggtccggt
gtttgcagac atgctttgaa aagctgtctc agtgagacat cagttatgtc caaaatgagt
ttaccttaga atcagaccgg ttttgccagg cgtcatgttt gcaaacatta tccacctaat
cagattttga aaggccggct ttcatgtcgc ctgcctgaga ctcataacag atccccatta
taagcgcgtt tacacagcaa aatgatttta ttgagaaaac cagcattaag tactgttgcc
ggctcagttt tccattgcat actatctact taaagtcccg ttctcatttg taagtgttcc
gatctttccc cacggagaaa actgagcaga gctgccgtgt cgcaggcttt ctgctgctga
tgtcgcatct ctttgctttc ccctccttag tccatactcc aagtaagtga actcagacta
ccagcaactt tttaactgaa aagtatctgt ccatgatgat caagatgcag ctcttcgtgt
ttttattttg tctttttttt tttttttttt ttttggaggg aaggagagac atcaactgga
caaaatgcaa aatttggatg tgggacaatt gctttttgga gacgtgaata gctgtactgt
acgtattatt ttgtgtggca tgctaacttt gagccgggca ctggcctaat aaagtctttg
taatcctccc agcaatccta taaagcagac gcagatagta aagattttag gctttgcagg
ccacgtacag actgtcacat gttctctgtt tttaaaagtg tcaacaacat tcttagctca
aaaacaggct gcaggtggga tggtgcccac aggccatagt tggctgaccc cggctagggt
gtaggcactt agcattccac tgtataaagg ggaaacccag gtcatactgc gtgtgcgtgg
gtgggaagcc ggatgtggaa tacaggtggt ccctgagtct ccaggaacca ctgagctcca
gctgttcaca cccacactct gcggcgcaag caactacctc gccacggttt agccttggtc
tagcagcgac tttaaccttg aatgttgcat ttctgaaaaa tttagaatct tgaaagtaaa
ggacgtccct ccggtgaata aaattaggcg caattataga atacatgtat tatggccacg
tagcaatgac tgtattaggg ctctgctagt tctgtaataa atagacccga aaagcaa
SEQ ID NO: 6
MAAAAVDSAMEVVPALAEEAAPEVAGLSCLVNLPGEVLEYILCC
GSLTAADIGRVSSTCRRLRELCQSSGKVWKEQFRVRWPSLMKHYSPTDYVNWLEEYKV
RQKAGLEARKIVASFSKRFFSEHVPCNGFSDIENLEGPEIFFEDELVCILNMEGRKAL
TWKYYAKKILYYLRQQKILNNLKAFLQQPDDYESYLEGAVYIDQYCNPLSDISLKDIQ
AQIDSIVELVCKTLRGINSRHPSLAFKAGESSMIMEIELQSQVLDAMNYVLYDQLKFK
GNRMDYYNALNLYMHQVLIRRTGIPISMSLLYLTIARQLGVPLEPVNFPSHFLLRWCQ
GAEGATLDIFDYIYIDAFGKGKQLTVKECEYLIGQHVTAALYGVVNVKKVLQRMVGNL
LSLGKREGIDQSYQLLRDSLDLYLAMYPDQVQLLLLQARLYFHLGIWPEKSFCLVLKV
LDILQHIQTLDPGQHGAVGYLVQHTLEHIERKKEEVGVEVKLRSDEKHRDVCYSIGLI
MKHKRYGYNCVIYGWDPTCMMGHEWIRNMNVHSLPHGHHQPFYNVLVEDGSCRYAAQE
NLEYNVEPQEISHPDVGRYFSEFTGTHYIPNAELEIRYPEDLEFVYETVQNIYSAKKE
NIDE
SEQ ID NO: 7
ctcttatata aattctaaaa attgatgttc taagaagaga ggtagaattt gaatgactgg
gttacttcct agactcttcc tccttctctt aagtacagta tagttctttc tctgaaaatc
ttcagtctct tagttccaga tgggttctct atggtaagaa tacaggacat gtagaaggcc
ctaggggaat gctttcttcc ccagatcttt gccctgtagt aggtttcagc tgagcaagga
cgagtagttt ttctggtgtt tggcctcctc tgttgggtgg aaaaagactt tcttctctat
tttcctagtt atatatgcta tcatatgtct gtttttctcc tcttgaagtt tccctgaaac
ctgggctctt gaagacgcat cactggagca gatggataat ggagactggg gctatatgat
gactgaccca gtcacattaa atgtaggtgg acacttgtat acaacgtctc tcaccacatt
gacgcgttac ccggattcca tgcttggagc tatgtttggg ggggacttcc ccacagctcg
agaccctcaa ggcaattact ttattgatcg agatggacct cttttccgat atgtcctcaa
cttcttaaga acttcagaat tgaccttacc gttggatttt aaggaatttg atctgcttcg
gaaagaagca gatttttacc agattgagcc cttgattcag tgtctcaatg atcctaagcc
tttgtatccc atggatactt ttgaagaagt tgtggagctg tctagtactc ggaagctttc
taagtactcc aacccagtgg ctgtcatcat aacgcaacta accatcacca ctaaggtcca
ttccttacta gaaggcatct caaattattt taccaagtgg aataagcaca tgatggacac
cagagactgc caggtttcct ttacttttgg accctgtgat tatcaccagg aagtttctct
tagggtccac ctgatggaat acattacaaa acaaggtttc acgatccgca acacccgggt
gcatcacatg agtgagcggg ccaatgaaaa cacagtggag cacaactgga ctttctgtag
gctagcccgg aagacagacg actgatctcc gaccctgcca caggttcctg gaaagactct
ccaggaaatg gaagatactg attttttttt ttaaatcaca gtgtgagata ttttttttct
tttaaatagt tgtatttatt tgaaggcagt gaggaccaga aggaagtttt gtgctttggc
agactcctcc atgttttgtt cccttccccc tgagtatgca tgtgcctgtt cagagtctcc
agataccttt tttataaaaa gaagtctgaa aatcattatg gtatataatc tacccttaac
agagcttttc ttattacagt gctaaaatga tttctgataa aatggtccct aactcaacta
gaaggctaaa aatacaagaa tgaaagaata agcagagtac tcatgatgcc tttgagaaaa
atcaaaacat catgtagggt gacctagttt ccaaaccaat aaataagtag tattgtaata
ttaaaggaaa actgttccaa tcatttaaaa gtacttatta agtactgctt tttacagtta
tgacaactgt ttctttctat gcatataaat caaggaacca aatatctgta gccatggaaa
tgtctgacta gaaatattta tattgaattc tgaatacaaa atgtccctgt ggtagaaaac
ttactcttta tgcctggtgc agtataattc ccaagtgtac tgtctaccag aaaaaaaaaa
caaaactaat aaaaaatgaa atatgaaaat taaaaaaaaa
SEQ ID NO: 8
MDNGDWGYMMTDPVTLNVGGHLYTTSLTTLTRYPDSMLGAMFGG
DFPTARDPQGNYFIDRDGPLFRYVLNFLRTSELTLPLDFKEFDLLRKEADFYQIEPLI
QCLNDPKPLYPMDTFEEVVELSSTRKLSKYSNPVAVIITQLTITTKVHSLLEGISNYF
TKWNKHMMDTRDCQVSFTFGPCDYHQEVSLRVHLMEYITKQGFTIRNTRVHHMSERAN
ENTVEHNWTFCRLARKTDD
SEQ ID NO: 9
gactattgcg cctgcgccag cgccggctgc gagactgggg ccgtggctgc tggtcccggg
tgatgctagg cggctccctg ggctccaggc tgttgcgggg tgtaggtggg agtcacggac
ggttcggggc ccgaggtgtc cgcgaaggtg gcgcagccat ggcggcaggg gagagcatgg
ctcagcggat ggtctgggtg gacctggaga tgacaggatt ggacattgag aaggaccaga
ttattgagat ggcctgtctg ataactgact ctgatctcaa cattttggct gaaggtccta
acctgattat aaaacaacca gatgagttgc tggacagcat gtcagattgg tgtaaggagc
atcacgggaa gtctggcctt accaaggcag tgaaggagag tacaattaca ttgcagcagg
cagagtatga atttctgtcc tttgtacgac agcagactcc tccagggctc tgtccacttg
caggaaattc agttcatgaa gataagaagt ttcttgacaa atacatgccc cagttcatga
aacatcttca ttatagaata attgatgtga gcactgttaa agaactgtgc agacgctggt
atccagaaga atatgaattt gcaccaaaga aggctgcttc tcatagggca cttgatgaca
ttagtgaaag catcaaagag cttcagtttt accgaaataa catcttcaag aaaaaaatag
atgaaaagaa gaggaaaatt atagaaaatg gggaaaatga gaagaccgtg agttgatgcc
agttatcatg ctgccactac atcgttatct ggaggcaact tctggtggtt tttttttctc
acgctgatgg cttggcagag caccttcggt taacttgcat ctccagattg attactcaag
cagacagcac acgaaatact atttttctcc taatatgctg tttccattat gacacagcag
ctcctttgta agtaccaggt catgtccatc ccttggtaca tatatgcatt tgcttttaaa
ccatttcttt tgtttaaata aataaataag taaataaagc tagttctatt gaaatgcaaa
SEQ ID NO: 10
MLGGSLGSRLLRGVGGSHGRFGARGVREGGAAMAAGESMAQRMV
WVDLEMTGLDIEKDQIIEMACLITDSDLNILAEGPNLIIKQPDELLDSMSDWCKEHHG
KSGLTKAVKESTITLQQAEYEFLSFVRQQTPPGLCPLAGNSVHEDKKFLDKYMPQFMK
HLHYRIIDVSTVKELCRRWYPEEYEFAPKKAASHRALDDISESIKELQFYRNNIFKKK
IDEKKRKIIENGENEKTVS
SEQ ID NO: 11
ctcaggactc agaggctggg atcatggtag atggaaccct ccttttactc ctctcggagg
ccctggccct tacccagacc tgggcgggct cccactcctt gaagtatttc cacacttccg
tgtcccggcc cggccgcggg gagccccgct tcatctctgt gggctacgtg gacgacaccc
agttcgtgcg cttcgacaac gacgccgcga gtccgaggat ggtgccgcgg gcgccgtgga
tggagcagga ggggtcagag tattgggacc gggagacacg gagcgccagg gacaccgcac
agattttccg agtgaatctg cggacgctgc gcggctacta caatcagagc gaggccgggt
ctcacaccct gcagtggatg catggctgcg agctggggcc cgacgggcgc ttcctccgcg
ggtatgaaca gttcgcctac gacggcaagg attatctcac cctgaatgag gacctgcgct
cctggaccgc ggtggacacg gcggctcaga tctccgagca aaagtcaaat gatgcctctg
aggcggagca ccagagagcc tacctggaag acacatgcgt ggagtggctc cacaaatacc
tggagaaggg gaaggagacg ctgcttcacc tggagccccc aaagacacac gtgactcacc
accccatctc tgaccatgag gccaccctga ggtgctgggc cctgggcttc taccctgcgg
agatcacact gacctggcag caggatgggg agggccatac ccaggacacg gagctcgtgg
agaccaggcc tgcaggggat ggaaccttcc agaagtgggc agctgtggtg gtgccttctg
gagaggagca gagatacacg tgccatgtgc agcatgaggg gctacccgag cccgtcaccc
tgagatggaa gccggcttcc cagcccacca tccccatcgt gggcatcatt gctggcctgg
ttctccttgg atctgtggtc tctggagctg tggttgctgc tgtgatatgg aggaagaaga
gctcaggtgg aaaaggaggg agctactcta aggctgagtg gagcgacagt gcccaggggt
ctgagtctca cagcttgtaa agcctgagac agctgccttg tgtgcgactg agatgcacag
ctgccttgtg tgcgactgag atgcaggatt tcctcacgcc tcccctatgt gtcttagggg
actctggctt ctctttttgc aagggcctct gaatctgtct gtgtccctgt tagcacaatg
tgaggaggta gagaaacagt ccacctctgt gtctaccatg acccccttcc tcacactgac
ctgtgttcct tccctgttct cttttctatt aaaaataaga acctgggcag agtgcggcag
ctcatgcctg taatcccagc acttagggag gccgaggagg gcagatcacg aggtcaggag
atcgaaacca tcctggctaa cacggtgaaa ccccgtctct actaaaaaat acaaaaaatt
agctgggcgc agaggcacgg gcctgtagtc ccagctactc aggaggcgga ggcaggagaa
tggcgtcaac ccgggaggcg gaggttgcag tgagccagga ttgtgcgact gcactccagc
ctgggtgaca gggtgaaacg ccatctcaaa aaataaaaat tgaaaaataa aaaaagaacc
tggatctcaa tttaattttt catattcttg caatgaaatg gacttgagga agctaagatc
atagctagaa atacagataa ttccacagca catctctagc aaatttagcc tattcctatt
ctctagccta ttccttacca cctgtaatct tgaccatata ccttggagtt gaatattgtt
ttcatactgc tgtggtttga atgttccctc caacactcat gttgagactt aatccctaat
gtggcaatac tgaaaggtgg ggcctttgag atgtgattgg atcgtaaggc tgtgccttca
ttcatgggtt aatggattaa tgggttatca caggaatggg actggtggct ttataagaag
aggaaaagag aactgagcta gcatgcccag cccacagaga gcctccacta gagtgatgct
aagtggaaat gtgaggtgca gctgccacag agggccccca ccagggaaat gtctagtgtc
tagtggatcc aggccacagg agagagtgcc ttgtggagcg ctgggagcag gacctgacca
ccaccaggac cccagaactg tggagtcagt ggcagcatgc agcgccccct tgggaaagct
ttaggcacca gcctgcaacc cattcgagca gccacgtagg ctgcacccag caaagccaca
ggcacggggc tacctgaggc cttgggggcc caatccctgc tccagtgtgt ccgtgaggca
gcacacgaag tcaaaagaga ttattctctt cccacagata ccttttctct cccatgaccc
tttaacagca tctgcttcat tcccctcacc ttcccaggct gatctgaggt aaactttgaa
gtaaaataaa agctgtgttt gagcatca
SEQ ID NO: 12
MVDGTLLLLLSEALALTQTWAGSHSLKYFHTSVSRPGRGEPRFI
SVGYVDDTQFVRFDNDAASPRMVPRAPWMEQEGSEYWDRETRSARDTAQIFRVNLRTL
RGYYNQSEAGSHTLQWMHGCELGPDGRFLRGYEQFAYDGKDYLTLNEDLRSWTAVDTA
AQISEQKSNDASEAEHQRAYLEDTCVEWLHKYLEKGKETLLHLEPPKTHVTHHPISDH
EATLRCWALGFYPAEITLTWQQDGEGHTQDTELVETRPAGDGTFQKWAAVVVPSGEEQ
RYTCHVQHEGLPEPVTLRWKPASQPTIPIVGIIAGLVLLGSVVSGAVVAAVIWRKKSS
GGKGGSYSKAEWSDSAQGSESHSL
SEQ ID NO: 13
gcgggcggcg cgagcgaggg gcagaggcga gagacgccgg cggggcgcgg gcgcggcggc
cccggaggat gctgctgagc cccggcactg cctggctgcg agcacatgat ggcgatacgg
gagctcaaag tgtgccttct cggggacact ggggttggga aatcaagcat cgtgtgtcga
tttgtccagg atcactttga ccacaacatc agccctacta ttggggcatc ttttatgacc
aaaactgtgc cttgtggaaa tgaacttcac aagttcctca tctgggacac tgctggtcag
gaacggtttc attcattggc tcccatgtac tatcgaggct cagctgcagc tgttatcgtg
tatgatatta ccaagcagga ttcattttat accttgaaga aatgggtcaa ggagctgaaa
gaacatggtc cagaaaacat tgtaatggcc atcgctggaa acaagtgcga cctctcagat
attagggagg ttcccctgaa ggatgctaag gaatacgctg aatccatagg tgccatcgtg
gttgagacaa gtgcaaaaaa tgctattaat atcgaagagc tctttcaagg aatcagccgc
cagatcccac ccttggaccc ccatgaaaat ggaaacaatg gaacaatcaa agttgagaag
ccaaccatgc aagccagccg ccggtgctgt tgacccaagg gccgtggtcc acggtacttg
aagaagccag agcccacatc ctgtgcactg ctgaaggacc ctacgctcgg tggcctggca
cctcactttg agaagagtga gcacactggc tttgcatcct ggaagacctg cagggggcgg
ggcaggaaat gtacctgaaa aggattttag aaaaccctgg gaaaacccac cacaccacca
caaaatggcc tttagtgtat gaaatgcaca tggaggggat gtagttgcat ttttgctaaa
aaaaaaaaaa aacctttaaa aattgttgga tgtgtacaaa agtcttactg ccttattatg
tgtatgggat tctaaagtgg cattccactt ggatttcctg tgctacctat ccaaattcca
gtaactactt cagtgtcatt gcctttgtta cctaaccaac cttcactgaa aggcaaattt
agttcaggag gttagttttt agctagcttt ggaagtaagc ctttatttat tactttttgg
aggaaatcag agaagtgtca atggaccgtc actcagactg agacttgagt tattacagaa
gccaggaaaa gtgtattaga aactgttgtc tacaccactt ttaattggtg aacaattttt
ctaagttatg gtcatatata cccaaacaaa ccaaatcaaa ctaaattact gcatataatt
ttgggattgg gtggcctagt ttgaaagagt gatttaagta atcactatgt aagtggtgag
agatgcagga catacacatt attcaagaga ccacctgaca tgcatctcct ccgcaggaat
acattcgtcc tctcttagag aagtttaacg cacatagtat tattttacta agagaatatc
tcttggtgtc atatctaggg gaagagaatt aactagaatt aaatttaatg tttgaatcta
aatcattggg caaacttcta ataataacaa ttaataatag gttacaggaa agccagccag
aggaagtgtc agcactttaa aattctagac cccaaaaaac tacaaaatca gaaaaagtat
ttttatgttt ctagcttgag gagaagggct ttagggctaa ccagaggtct gaccctagaa
tgccaaggaa ctgagaatgg gctccgatga aaaccttcct tttcagattc cctgtctgct
caattaaaga tgtttgaatc caaaggaagt caaggaagaa aaagcatgga aaggaagaga
actgattcct actgaaaatt caaattctat taccattcta actttcataa aaagttggga
tcaagaagca gctgatttcc tgccagggct tatattaggg ggtgattctt aaaggacatt
aggattggtg ctcagaaatg gttaatcatg ctgtgtgcta gccagggcca gctggtacct
tctttgccat gagcattcaa gggacggcta acctttattg acaatctata tcgcaaaagt
caggaaagag gttgtgagct gattggatta aagacctggc acttcagtaa ctcagcacgc
ttccacttca ctcaacttaa gagagttcat tgacagtgtt aggatgtgaa ggctgggaaa
cacttatttt gcttcaagag ttccacttgg ctctcccaaa taggtacctc aaaaactgtt
agcaagcggc atttggatgt cttgacaggg gctttgcagg gatttttagg gttttttcca
cattgtccac attaatggtt ggcatgattg tgcttgcagg ccaagaaatg atcatacccc
ttgccaaagg taaaaaaaaa aaaaaaaaaa tgagttgaaa attgaagtga cctctttcca
gctgagttgc aggcttattt tgtaaccttt cctcatccag ttttccctga gaacctgggt
ttatctctag atagctgttc aggtttttta gctgaggggt aagtatccta gctgagagtt
ttgcatcttt gggctgggtt tgcagtggtt gtgttttgca taaaatgtct agtctttgcc
acagatagtg agctacccac taatgagccc atggttttat ttcagaagca catgagggtg
tgaaaccact ctgttacctt tctgtattgt cttagctatt caagccagtc agaggataat
atatatattc tcatcagcac tcagagtagt cagtgaagag agtagatcac acttgggcac
accaggattc acataaacat tgtatcttct ctgtggatgc tcaggccttg tctacaatga
ggctttacaa ccttcctttg ttttggctcg ggattacttc ctggctgtct aataattgaa
ccataaccat gtaatattat gtaaaggcct ggaaattact gttgctaaaa aaagtcatgt
agtttcatgt agtgtagcat ccttggcatc gttttccaaa atttgttcct tctccctttt
ttttttcttt cgtgtgtggc atgagtgtgt atctgtgtaa atatgattgt atatgtgtta
ctccgatatg taatccattt cactggctga gtttggcccc tagccatgtg ttaatataaa
gtaggcatgg cttcccaatg gaaatctctg agaatgacag tggagttgtg caagcatttt
acattgccac ataattgact tgccatttta tggttaaaaa cggcacatta ggcagttgaa
tatgacgtta ccttgcagac taaaaggttg aaggcccgaa actaactttt agctaacaat
aagggctgtg ccccaatgga aactgagttc attttctgag aaaggtttgg atgactgaaa
tatttcctct acagtcaagg actttggcat gtggtggctg aaactgagct tttttgtgtg
ggctccagtt ctcactgttc tgcaatgctc atggcaagtt gaatggtgag ctagcttata
aattaaagag ctctgaactg tattcagacc gactgggtat ctagcttact gttttaacat
cattgttgaa accagaccct gtagtccagt ggtgctgccc tgttgtgcaa actgctcctt
tttctcgtgt ttttgtaaag agcttccatc tgggctggac ccagttcttg cacatacaag
acaccgctgc agtcagctag gacctttccg ccatgtattc tattctgtag taaagcattt
ccatcaacaa tgcctaattg tatctgttat ttttggttta acacacactg attcatacta
ataaatattt tcagtttta
SEQ ID NO: 14
MMAIRELKVCLLGDTGVGKSSIVCRFVQDHFDHNISPTIGASFM
TKTVPCGNELHKFLIWDTAGQERFHSLAPMYYRGSAAAVIVYDITKQDSFYTLKKWVK
ELKEHGPENIVMAIAGNKCDLSDIREVPLKDAKEYAESIGAIVVETSAKNAINIEELF
QGISRQIPPLDPHENGNNGTIKVEKPTMQASRRCC
SEQ ID NO: 15
ctctcaacca tcaggttcgg cagcccgcgg cgccgcctgg cagctcctcc tcttctccgc
cccgccggcc gcgggcgcgg gggacgtcag cgctgccagc gtggaaggag ctgcggggcg
cgggaggagg aagtagagcc cgggaccgcc aggccaccac cggccgcctc agccatggac
gcgtccctgg agaagatagc agaccccacg ttagctgaaa tgggaaaaaa cttgaaggag
gcagtgaaga tgctggagga cagtcagaga agaacagaag aggaaaatgg aaagaagctc
atatccggag atattccagg cccactccag ggcagtgggc aagatatggt gagcatcctc
cagttagttc agaatctcat gcatggagat gaagatgagg agccccagag ccccagaatc
caaaatattg gagaacaagg tcatatggct ttgttgggac atagtctggg agcttatatt
tcaactctgg acaaagagaa gctgagaaaa cttacaacta ggatactttc agataccacc
ttatggctat gcagaatttt cagatatgaa aatgggtgtg cttatttcca cgaagaggaa
agagaaggac ttgcaaagat atgtaggctt gccattcatt ctcgatatga agacttcgta
gtggatggct tcaatgtgtt atataacaag aagcctgtca tatatcttag tgctgctgct
agacctggcc tgggccaata cctttgtaat cagctcggct tgcccttccc ctgcttgtgc
cgtgtaccct gtaacactgt gtttggatcc cagcatcaga tggatgttgc cttcctggag
aaactgatta aagatgatat agagcgagga agactgcccc tgttgcttgt cgcaaatgca
ggaacggcag cagtaggaca cacagacaag attgggagat tgaaagaact ctgtgagcag
tatggcatat ggcttcatgt ggagggtgtg aatctggcaa cattggctct gggttatgtc
tcctcatcag tgctggctgc agccaaatgt gatagcatga cgatgactcc tggcccgtgg
ctgggtttgc cagctgttcc tgcggtgaca ctgtataaac acgatgaccc tgccttgact
ttagttgctg gtcttacatc aaataagccc acagacaaac tccgtgccct gcctctgtgg
ttatctttac aatacttggg acttgatggg tttgtggaga ggatcaagca tgcctgtcaa
ctgagtcaac ggttgcagga aagtttgaag aaagtgaatt acatcaaaat cttggtggaa
gatgagctca gctccccagt ggtggtgttc agatttttcc aggaattacc aggctcagat
ccggtgttta aagccgtccc agtgcccaac atgacacctt caggagtcgg ccgggagagg
cactcgtgtg acgcgctgaa tcgctggctg ggagaacagc tgaagcagct ggtgcctgca
agcggcctca cagtcatgga tctggaagct gagggcacgt gtttgcggtt cagccctttg
atgaccgcag cagttttagg aactcgggga gaggatgtgg atcagctcgt agcctgcata
gaaagcaaac tgccagtgct gtgctgtacg ctccagttgc gtgaagagtt caagcaggaa
gtggaagcaa cagcaggtct cctatatgtt gatgacccta actggtctgg aataggggtt
gtcaggtatg aacatgctaa tgatgataag agcagtttga aatcagatcc cgaaggggaa
aacatccatg ctggactcct gaagaagtta aatgaactgg aatctgacct aacctttaaa
ataggccctg agtataagag catgaagagc tgcctttatg tcggcatggc gagcgacaac
gtcgatgctg ctgagctcgt ggagaccatt gcggccacag cccgggagat agaggagaac
tcgaggcttc tggaaaacat gacagaagtg gttcggaaag gcattcagga agctcaagtg
gagctgcaga aggcaagtga agaacggctt ctggaagagg gggtgttgcg gcagatccct
gtagtgggct ccgtgctgaa ttggttttct ccggtccagg ctttacagaa gggaagaact
tttaacttga cagcaggctc tctggagtcc acagaaccca tatatgtcta caaagcacaa
ggtgcaggag tcacgctgcc tccaacgccc tcgggcagtc gcaccaagca gaggcttcca
ggccagaagc cttttaaaag gtccctgcga ggttcagatg ctttgagtga gaccagctca
gtcagtcaca ttgaagactt agaaaaggtg gagcgcctat ccagtgggcc ggagcagatc
accctcgagg ccagcagcac tgagggacac ccaggggctc ccagccctca gcacaccgac
cagaccgagg ccttccagaa aggggtccca cacccagaag atgaccactc acaggtagaa
ggaccggaga gcttaagatg agactcattg tgtggtttga gactgtactg agtattgttt
cagggaagat gaagttctat tggaaatgtg aactgtgcca catactaata taaattactg
ttgtttgtgc ttcactggga ttttggcaca aatatgtgcc tgaaaggtag gctttctagg
aggggagtca gcttgtctaa cttcatgtac atgtagaacc acgtttgctg tcctactacg
acttttccct aagttaccat aaacacattt tattcacaaa aaacacttcg aatttcaagt
gtctaccagt agcacccttg ctctttctaa acataagcct aagtatatga ggttgcccgt
ggcaactttt tggtaaaaca gcttttcatt agcactctcc aggttctctg caacacttca
cagaggcgag actggctgta tcctttgctg tcggtcttta gtacgatcaa gttgcaatat
acagtgggac tgctagactt gaaggagagc agtgattgtg ggattgtaaa taagagcatc
agaagccctc cccagctact gctcttcgtg gagacttagt aaggactgtg tctacttgag
ctgtggcaag gctgctgtct gggactgtcc tctgccacaa ggccatttct cccattatat
accgtttgta aagagaaact gtaaagtctc ctcctgacca tatattttta aatactggca
aagcttttaa aattggcaca caagtacaga ctgtgctcat ttctgtttag tatctgaaaa
cctgatagat gctaccctta agagcttgct cttccgtgtg ctacgtagca cccacctggt
taaaatctga aaacaagtac ccctttgacc tgtctcccac tgaagcttct actgccctgg
cagctcgcct gggcccaact cagaaacagg agccagcaga gcactctctc acgctgatcc
agccgggcac cctgcttaag tcagtagaag ctcgctggca ctgcccgttc ctacttttcc
gaagtactgc gtcactttgt cgtaagtaat ggcccctgtg ccttcttaat ccagcagtca
agcttttggg agacctgaaa atgggaaaat tcacactggg tttctggact gtagtattgg
aagccttagt tatagtatat taagcctata attatactct gatttgatgg gatttttgac
atttacactt gtcaaaatgc agggggtttt ttttggtgca gatgattaaa cagtcttccc
tatttggtgc aatgaagtat agcagataaa atgggggagg ggtaaattat caccttcaag
aaaattacat gtttttatat atatttggaa ttgttaaatt ggttttgctg aaacatttca
cccttgagat attatttgaa tgttggtttc aataaaggtt cttgaaattg ttaccagtga
attcagttta taaatcttat tacaaaagac ttacccacgt acctgaaata gctgccgata
gaccagtgag aggtaggttc tcctctgccc gttattaccg accaaaaaaa aaactggaca
tcaatttttt agtaaaccaa aaaataagtc tcaacaaatg cctttgccaa aataaggttt
tattttgaaa gtcatttgat gaaagtcatt tgaaagacac tgaggaggga aggaggccta
agacccaaca gatgtaggat ccagatctgg attcgtgcca gccccaccaa tggtctgtca
ggccaagaag gtgctttctt tggtaattca tgttttttaa cttcctggag aagagatctt
ttcccacaag ccatcttcat tttttttgta gagtagggct ttatttccag aaaacagtgt
gtgagctgga gatgggtgtt tttttaaaaa catcaaggta gatctaatat gttcaacaaa
gtggggtggc tcagccagag gcgaagtgga aagattctga aaacacaaga tggtgggcat
tagagaagcc aaccttactg tcccctgctg tgataaagat gtcaaagtat ctttgttctt
ggacacaaat atatataata aaatacgtta agaaatga
SEQ ID NO: 16
MDASLEKIADPTLAEMGKNLKEAVKMLEDSQRRTEEENGKKLIS
GDIPGPLQGSGQDMVSILQLVQNLMHGDEDEEPQSPRIQNIGEQGHMALLGHSLGAYI
STLDKEKLRKLTTRILSDTTLWLCRIFRYENGCAYFHEEEREGLAKICRLAIHSRYED
FVVDGFNVLYNKKPVIYLSAAARPGLGQYLCNQLGLPFPCLCRVPCNTVFGSQHQMDV
AFLEKLIKDDIERGRLPLLLVANAGTAAVGHTDKIGRLKELCEQYGIWLHVEGVNLAT
LALGYVSSSVLAAAKCDSMTMTPGPWLGLPAVPAVTLYKHDDPALTLVAGLTSNKPTD
KLRALPLWLSLQYLGLDGFVERIKHACQLSQRLQESLKKVNYIKILVEDELSSPVVVF
RFFQELPGSDPVFKAVPVPNMTPSGVGRERHSCDALNRWLGEQLKQLVPASGLTVMDL
EAEGTCLRFSPLMTAAVLGTRGEDVDQLVACIESKLPVLCCTLQLREEFKQEVEATAG
LLYVDDPNWSGIGVVRYEHANDDKSSLKSDPEGENIHAGLLKKLNELESDLTFKIGPE
YKSMKSCLYVGMASDNVDAAELVETIAATAREIEENSRLLENMTEVVRKGIQEAQVEL
QKASEERLLEEGVLRQIPVVGSVLNWFSPVQALQKGRTFNLTAGSLESTEPIYVYKAQ
GAGVTLPPTPSGSRTKQRLPGQKPFKRSLRGSDALSETSSVSHIEDLEKVERLSSGPE
QITLEASSTEGHPGAPSPQHTDQTEAFQKGVPHPEDDHSQVEGPESLR

What is claimed is:

1. A method for treating acute rejection (AR) of a transplant in a subject comprising:
(a) measuring expression levels of two or more genes selected from the group consisting of DIP2C, ENOSF1, FBXO21, KCTD6, PDXDC1, REXO2, HLA-E, and RAB31 in a biological sample from a subject having an organ transplant, wherein differential expression of two or more genes selected from the group consisting of DIP2C, ENOSF1, FBXO21, KCTD6, PDXDC1, REXO2, HLA-E, and RAB31, as compared to control, indicates the subject has an increased likelihood of AR of the transplant; and
(b) administering an effective amount of corticosteroid or antibody therapy to the subject.

2. The method of claim 2, wherein the expression levels of DIP2C, ENOSF1, FBXO21, KCTD6, PDXDC1, REXO2, HLA-E, and RAB31 are measured.

3. The method of claim 1, wherein differential expression results in a genetic signature wherein DIP2C, ENOSF1, FBXO21, KCTD6, PDXDC1 and REXO2 are downregulated, and wherein HLA-E and RAB31 are upregulated.

4. The method of claim 1, wherein mRNA expression levels are measured.

5. The method of claim 1, wherein protein expression levels are measured.

6. The method of claim 1, wherein the acute cellular rejection is T cell-mediated rejection (TCMR) or antibody-mediated rejection (ABMR).

7. The method of claim 1, wherein the transplant is a kidney transplant.

8. The method of claim 1, wherein the subject is an adult.

9. The method of claim 1, wherein the subject is a pediatric subject.

10. The method of claim 1, wherein the antibody is selected from the group consisting of a lymphocyte-depleting antibody, an anti-thymoglobulin antibody, an anti-CD52 antibody, and an anti-CD3 antibody.

11. The method of claim 1, further comprising performing a biopsy on transplant tissue from the subject.

12. The method of claim 11, further comprising, if the biopsy shows antibody-mediated damage in the subject, administering plasma exchange therapy, intravenous immunoglobulin (Ig) therapy or a proteosomal inhibitor to the subject.

13. The method of claim 12, wherein the intravenous Ig therapy is administered in combination with rituximab.

14. A kit comprising:
(a) primers or probes for detection of two or more genes selected from the group consisting of DIP2C, ENOSF1, FBXO21, KCTD6, PDXDC1, REXO2, HLA-E, and RAB31.
